(19) United States
(12) Patent Application Publication
(10) Pub. No.: US 2020/0398075 A1
(43) Pub. Date: Dec. 24, 2020

(54) PHOTOBIOMODULATION THERAPY SYSTEMS AND METHODS

(71) Applicant: Joovv, Inc., San Clemente, CA (US)

(72) Inventors: Gregory Pfiffner, San Clemente, CA (US); Jakob Kishon, San Clemente, CA (US)

(21) Appl. No.: 16/904,243

(22) Filed: Jun. 17, 2020

Related U.S. Application Data
(60) Provisional application No. 62/863,247, filed on Jun. 18, 2019.
(60) Provisional application No. 62/872,835, filed on Jul. 11, 2019.

(51) Int. Cl. A61N 5/06

(52) U.S. Cl. CPC A61N 5/0616; A61N 2005/063; A61N 2005/0626; A61N 2005/0632; A61N 2005/0664; A61N 2005/0652; A61N 2005/0659; A61N 2005/0663; A61N 2005/064

(57) ABSTRACT

The disclosure includes a light therapy system comprising at least one light therapy panel, wherein each light therapy panel of the at least one light therapy panel comprises a first housing and a first plurality of lights arranged and configured to emit at least one of red light and infrared light. In some embodiments, the light therapy system further includes a console hub communicatively and electrically coupled to the at least one light therapy panel, the console hub configured to operate the at least one light therapy panel. The system may also include a remote control unit communicatively coupled to the console hub, wherein the remote control unit is configured to operate at least one of the console hub and the at least one light therapy panel.

### CROSS-REFERENCE TO RELATED APPLICATIONS

This application claims the benefit and is a non-provisional of U.S. Provisional Patent Application No. 62/872,835; filed Jul. 11, 2019; and entitled PHOTOBIOMODULATION THERAPY SYSTEMS AND METHODS; the entire contents of which are incorporated herein. This application also claims the benefit and is a non-provisional of U.S. Provisional Patent Application No. 62/863,247; filed Jun. 18, 2019; and entitled PHOTOBIOMODULATION THERAPY SYSTEMS AND METHODS; the entire contents of which are incorporated herein by reference.

### BACKGROUND

### Field

Various embodiments disclosed herein relate to photobiomodulation therapy systems and methods. Certain embodiments relate to photobiomodulation therapy systems having a curved appearance.

### Background

Photobiomodulation therapy (or light therapy) is a therapeutic technique that uses low-level wavelengths of light to improve health and treat a variety of health conditions, including skin issues, such as wrinkles, scars, and persistent wounds, among many other conditions. Photobiomodulation therapy uses non-ionizing light sources, including lasers, light emitting diodes, and/or broadband light, in the visible (400-700 nm) and infrared (700-1100 nm) electromagnetic spectrum. It is a nonthermal process involving endogenous chromophores eliciting photophysical (i.e. linear and nonlinear) and photochemical events at various biological scales. Similar to how plants use sunlight to heal and grow, humans and animals are able to harness these wavelengths of light and turn them into cellular energy. This treatment stimulates the body's natural healing processes.

Currently, there are a number of photobiomodulation therapy devices available on the market. However, many of these devices are too small and require multiple sessions to treat large areas. As a result, there is a need for a photobiomodulation therapy system that can treat several and/or larger areas in fewer treatments.

### SUMMARY

This disclosure includes a light therapy system comprising at least one light therapy panel, wherein each light therapy panel of the at least one light therapy panel comprises a first housing and a first plurality of lights arranged and configured to emit at least one of red light and infrared light, a console hub communicatively and electrically coupled to the at least one light therapy panel, the console hub configured to operate the at least one light therapy panel, and a remote control unit communicatively coupled to the console hub, wherein the remote control unit is configured to operate at least one of the console hub and the at least one light therapy panel.

In some embodiments, a first light therapy panel of the at least one light therapy panel comprises at least one light therapy unit and at least one light therapy pallet, wherein each light therapy unit of the at least one light therapy unit is elongate along a first direction, and wherein each light therapy pallet of the at least one light therapy pallet extends along a second direction that is perpendicular to the first direction. A first light therapy pallet of the at least one light therapy pallet may comprise a first light therapy unit, a second light therapy unit, and a third light therapy unit aligned along the second direction, wherein the first light therapy unit is located between the second light therapy unit and the third light therapy unit, and wherein the first light therapy unit, the second light therapy unit, and the third light therapy unit are electrically coupled.

In some embodiments, the first housing defines a curvature such that the first light therapy pallet curves along a third direction that is perpendicular to both the first direction and the second direction. The light therapy system may further comprise a first heat sink mechanically coupled to the first light therapy unit, the second light therapy unit, and the third light therapy unit, wherein the first heat sink is at least partially located within the first housing.

In some embodiments, the light therapy system further comprises a fourth light therapy unit, a fifth light therapy unit, a sixth light therapy unit, a seventh light therapy unit, a second heat sink, and a third heat sink, wherein the second heat sink is mechanically coupled to the fourth light therapy unit and the sixth light therapy unit and the third heat sink is mechanically coupled to the fifth light therapy unit and the seventh light therapy unit, and wherein the second heat sink and the third heat sink are at least partially located within the first housing.

The first light therapy unit may comprise a controller board and a board-to-board connector capable of electrically and communicatively coupling with another controller board. In some embodiments, the first light therapy panel comprises at least seven light therapy units, wherein the at least seven light therapy units are aligned along at least one of the first direction and the second direction. However, in other embodiments, the light therapy panel comprises six or fewer light therapy units aligned along at least one of the first direction and the second direction. In other embodiments, the light therapy panel comprises eight or more light therapy units aligned along at least one of the first direction and the second direction. In some embodiments, the first light therapy panel comprises at least five light therapy pallets, wherein the at least five light therapy pallets may be aligned along at least one of the first direction and the second direction. However, in other embodiments, the light therapy panel comprises four or fewer light therapy pallets aligned along at least one of the first direction and the second direction.

In some embodiments, each light therapy unit of the at least one light therapy unit comprises at least one LED cluster comprising six or more light emitting sources. The at least one LED cluster may comprise a first light emitting source capable of emitting royal blue light within a wavelength of approximately 380 nm to 505 nm, a second light emitting source capable of emitting blue light within a wavelength of approximately 410 nm to 530 nm, a third light emitting source capable of emitting green light within a wavelength of approximately 470 nm to 600 nm, a fourth light emitting source capable of emitting amber light within a wavelength of approximately 545 nm to 660 nm, a fifth light emitting source capable of emitting red light within a wavelength of approximately 590 nm to 720 nm, and a sixth light emitting source capable of emitting NIR light within a wavelength of approximately 725 nm to 900 nm.

In some embodiments, the at least one LED cluster comprises a first light emitting source capable of emitting royal blue light within a wavelength of approximately 450 nm to 460 nm, a second light emitting source capable of emitting blue light within a wavelength of approximately 470 nm to 485 nm, a third light emitting source capable of emitting green light within a wavelength of approximately 515 nm to 535 nm, a fourth light emitting source capable of emitting amber light within a wavelength of approximately 590 nm to 600 nm, a fifth light emitting source capable of emitting red (or crimson) light within a wavelength of approximately 650 nm to 670 nm, and a sixth light emitting source capable of emitting NIR light within a wavelength of approximately 840 nm to 860 nm. In some embodiments, the at least one LED cluster comprises a first light emitting source capable of emitting royal blue light having a typical wavelength of 455 nm, a second light emitting source capable of emitting blue light having a typical wavelength of approximately 475 nm, a third light emitting source capable of emitting green light having a typical wavelength of approximately 525 nm, a fourth light emitting source capable of emitting amber light having a typical wavelength of approximately 595 nm, a fifth light emitting source capable of emitting red (or crimson) light having a typical wavelength of approximately 660 nm, and a sixth light emitting source capable of emitting NIR light having a typical wavelength of approximately 850 nm. It should be appreciated that any of these light emitting sources may be used alone or in any combination with other light emitting sources. For example, in some embodiments, the at least one LED cluster comprises a light emitting source capable of emitting green light. While, in some embodiments, the at least one LED cluster comprises a first light emitting source capable of emitting blue light and a second light emitting source capable of emitting red (or crimson) light.

In some embodiments, each light therapy unit of the at least one light therapy unit comprises a first LED cluster, a second LED cluster, a third LED cluster, and a fourth LED cluster, wherein each of the first LED cluster, the second LED cluster, the third LED cluster, and the fourth LED cluster are aligned along the first direction.

Each light therapy unit of the at least one light therapy unit may further comprise a lens located over a single LED cluster of the at least one LED cluster. In some embodiments, the lens comprises a Fresnel lens. In some embodiments, the light therapy system comprises four LED clusters and four Fresnel lenses, such that each LED cluster of the four LED clusters is configured to emit light through a single Fresnel lens of the four Fresnel lenses. While Fresnel lenses are disclosed herein, other embodiments may use any type of lens capable of focusing light in a concentrated area.

In some embodiments, the console hub further comprises a liquid crystal display (LCD) touch screen arranged and configured to receive commands to operate the console hub. The light therapy system may comprise a second light therapy panel communicatively and electrically coupled to the console hub, the second light therapy panel comprising a second housing and a second plurality of lights arranged and configured to emit at least one of red light and infrared light. In some embodiments, when the first light therapy panel is located adjacent the second light therapy panel, the first light therapy panel and the second light therapy panel may be configured to curve at least about 90-degrees, or any curvature less than or greater than about 90-degrees.

The light therapy system may further comprise a third light therapy panel communicatively and electrically coupled to the console hub, the third light therapy panel comprising a third housing and a third plurality of lights arranged and configured to emit at least one of red light and infrared light. In some embodiments, the light therapy system also comprises a fourth light therapy panel communicatively and electrically coupled to the console hub, the fourth light therapy panel comprising a fourth housing and a fourth plurality of lights arranged and configured to emit at least one of red light and infrared light. By adding a third light therapy panel this may allow the system to curve more than 90-degrees, in some embodiments, the total curvature of the system with the third light therapy panel is about 135-degrees.

When the first light therapy panel is located adjacent the second light therapy panel, the second light therapy panel is located adjacent the third light therapy panel, and the third light therapy panel is located adjacent the fourth light therapy panel, the first light therapy panel, second light therapy panel, third light therapy panel, and fourth light therapy panel may be configured to curve at least about 180-degrees. In some embodiments, the light therapy system further comprises a console hub extender communicatively and electrically coupled to the console hub and at least one additional light therapy panel, such that the console hub extender enables the inclusion of at least five light therapy panels in the light therapy system.

### BRIEF DESCRIPTION OF THE DRAWINGS

These and other features, aspects, and advantages are described below with reference to the drawings, which are intended to illustrate, but not to limit, the invention. In the drawings, like reference characters denote corresponding features consistently throughout similar embodiments.

FIG. 1 illustrates a perspective view of a user using a light therapy system, according to some embodiments.

FIGS. 2A and 2B illustrate perspective views of a light therapy system including a console hub and multiple light therapy panels, according to some embodiments.

FIGS. 3A, 3B, 3C, 3D, 3E and 3F illustrate perspective views of a first light therapy panel of a light therapy system, according to some embodiments.

FIG. 4A illustrates a front isometric view of a first light therapy unit of a light therapy system, according to some embodiments.

FIG. 4B illustrates an exploded view of a first light therapy unit of a light therapy system, according to some embodiments.

FIGS. 4C and 4D illustrate front and back views, respectively, of a first light therapy unit of a light therapy system, according to some embodiments.

FIG. 4E illustrates a side view of a first light therapy unit of a light therapy system, according to some embodiments.

FIGS. 5A, 5B, and 5C illustrate perspective views of electrical components of a light therapy panel of a light therapy system, according to some embodiments.

FIGS. 5D, 5E, and 5F illustrate front and back views, respectively, of a light therapy pallet of a light therapy system, according to some embodiments.

FIG. 5G illustrates a schematic of the electrical components of a light therapy system, according to some embodiments.

FIGS. 6A and 6D illustrate graphs of the output intensity of lights of different wavelengths, according to some embodiments.

FIG. 6B illustrates a table of wavelengths for different colors of light, according to some embodiments.

FIGS. 6C, 6E, and 6F illustrate views of an LED cluster, according to some embodiments.

FIG. 7A illustrates a top view of a light therapy pallet including three heat sinks, according to some embodiments.

FIGS. 7B and 7C illustrate top views of a light therapy pallet including a single heat plate, according to some embodiments.

FIGS. 8A, 8B, and 8C illustrate top views of a plurality of curved light therapy panels, according to some embodiments.

FIG. 9A illustrates an isometric view of a console hub of a light therapy system, according to some embodiments.

FIGS. 9B and 9C illustrate front and back views, respectively, of a console hub of a light therapy system, according to some embodiments.

FIG. 10 illustrates a front view of a console hub extender of a light therapy system, according to some embodiments.

FIG. 11 illustrates a perspective view of a light therapy system including a console hub, a console hub extender, and eight light therapy panels, according to some embodiments.

### DETAILED DESCRIPTION

Although certain embodiments and examples are disclosed below, inventive subject matter extends beyond the specifically disclosed embodiments to other alternative embodiments and/or uses, and to modifications and equivalents thereof. Thus, the scope of the claims appended hereto is not limited by any of the particular embodiments described below. For example, in any system or method disclosed herein, the acts or operations of the system or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components.

For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

The terms “patient” and “user” may be used interchangeably. In addition, the terms “row of light therapy units” and “light therapy pallet” may be used interchangeably. Further, the terms “light therapy device” and “light therapy panel” may be used interchangeably.

### List of Reference Numbers

10—light therapy system

11—at least one light therapy unit

12—at least one light therapy panel

13—housing

14—light box/lens holder

18—console hub

20—panel cable

22—remote control unit

24—console extender

26—red light emitting diode (“LED”)

28—infrared LED

30—LED lens

31—Fresnel lens

32—treatment surface

36—plurality of connection ports

38—LCD touch screen

40—at least one fan

42—at least one ventilation opening

44—even distribution of red LEDs and infrared LEDs

52—driver board

53—controller board

54—fan

56—printed circuit board

58—pallet

60—heat sink

61—heat plate

62—at least one connection port

64—user

66—mounting screws

70—light therapy panel cable connection

72—back bucket

74—mounting hole

84—at least one flexible cable

86—board-to-board connector

88—LED cluster

90—curvature

Photobiomodulation therapy, also referred to as light therapy, provides an alternative option for treating many common ailments and diseases, and is commonly used to improve general health by promoting tissue repair, decreasing pain, and reducing inflammation. For example, when the human body is exposed to red light, blue light, green light, and/or near infrared light, subjects can expect to see improvement in multiple skin conditions, weight loss, muscle recovery, sexual performance, joint pain, and thyroid function. Instead of using prescription medications to solve these many problems, light therapy can be used in place of these traditional remedies to achieve safe and effective results.

Many top professionals have adopted light therapy, but oftentimes the light therapy devices and systems used are not big enough to treat an entire body at once. Accordingly, many embodiments described herein enable two or more light therapy devices to be coupled together in various ways. In doing so, the area of treatment can be expanded to reduce the time and number of treatments to achieve the desired results. As an additional improvement to existing light therapy devices, the light therapy devices disclosed herein comprise curved light panels which focus the wavelengths of emitted light to create a concentrated light field that brings more effective treatment to a user.

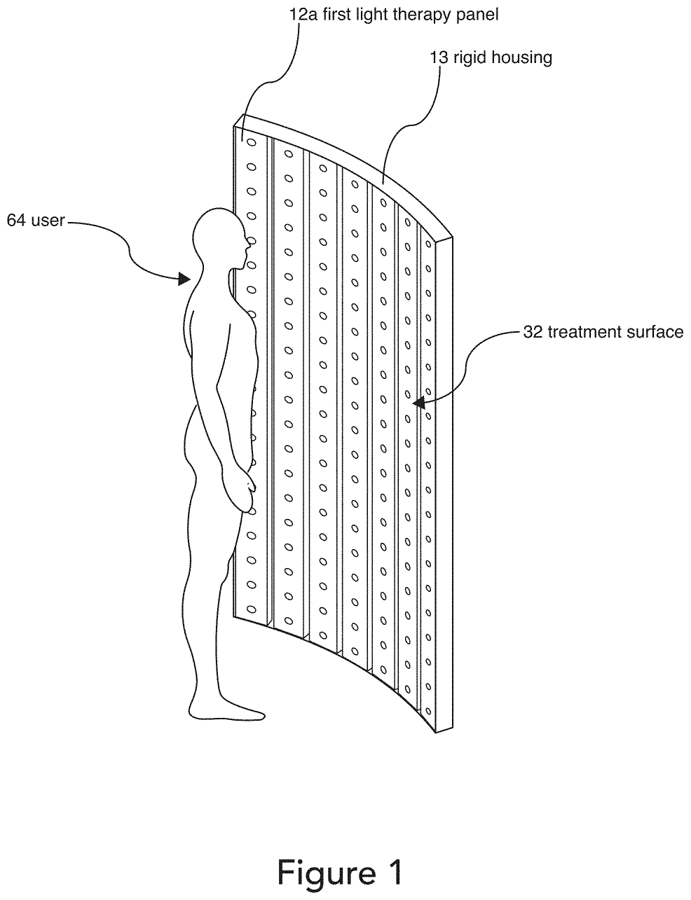

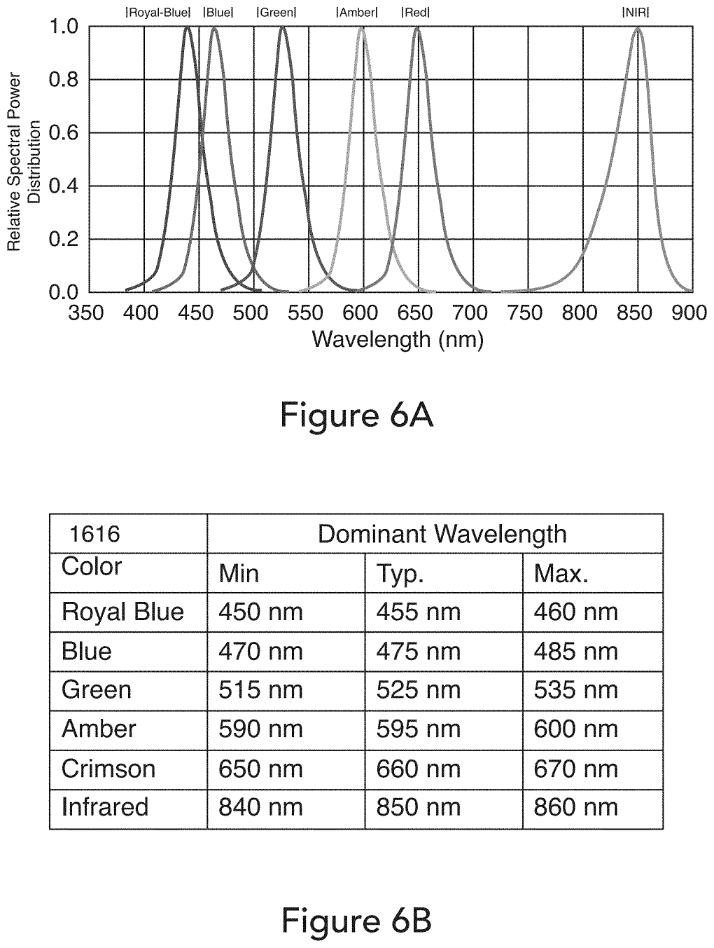

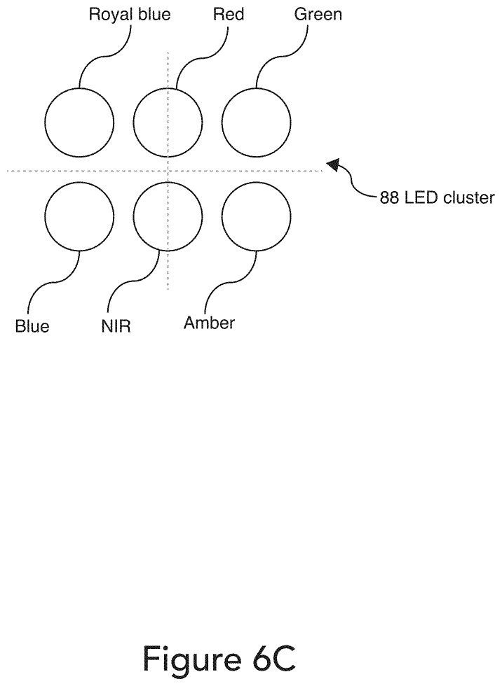

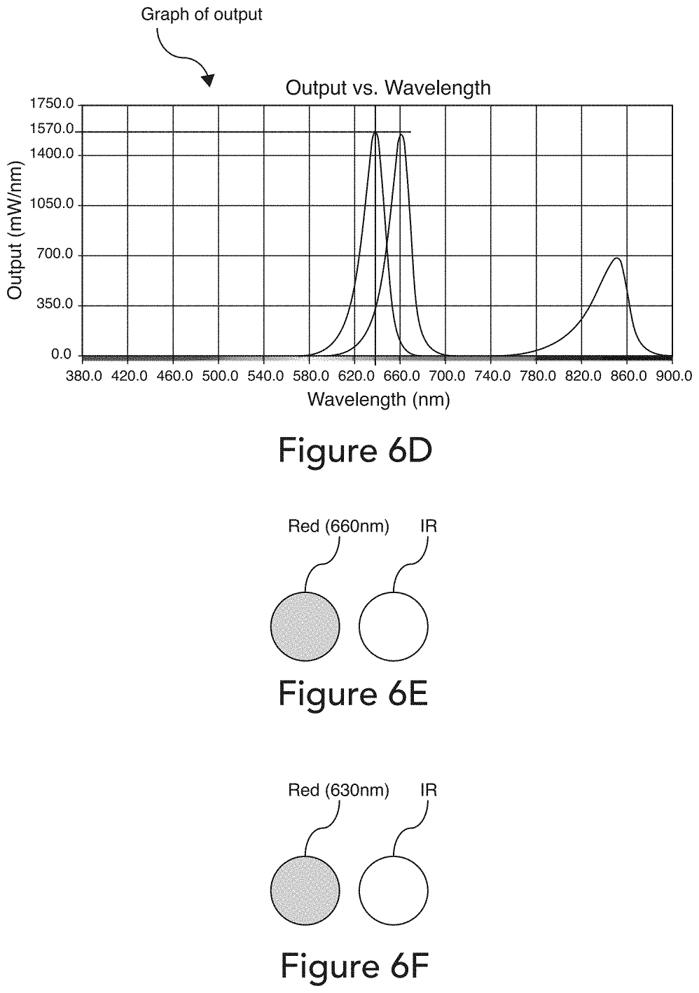

The disclosure includes a light therapy system 10 that includes at least one light therapy panel 12. As shown in FIG. 1, the light therapy system 10 may include a first light therapy panel 12a configured to emit light from a treatment surface 32 of a housing 13, in front of which a user 64 is positioned. In many embodiments, the first light therapy panel 12a emits at least one of red light, infrared (“IR”) light, and near infrared (“NIR”) light. The first light therapy panel 12a may also be configured to emit blue light, green, amber light, and light of other wavelengths. Further details of the emitted light will be discussed later in the disclosure, in particular with reference to FIGS. 6A-6F.

As illustrated in FIG. 1, the first light therapy panel 12a may define a curvature such that the first light therapy panel 12a at least partially curves around the user 64. The curvature may also result in better light distribution than a non-curved light therapy panel 12. In some embodiments, the at least one light therapy panel 12 may be sized such that it is approximately the same height as a user 64, as demonstrated in FIG. 1. In some embodiments, the at least one light therapy panel 12 is significantly smaller than shown in FIG. 1. For example, the at least one light therapy panel 12 may measure about 12″ wide by 36″ tall.

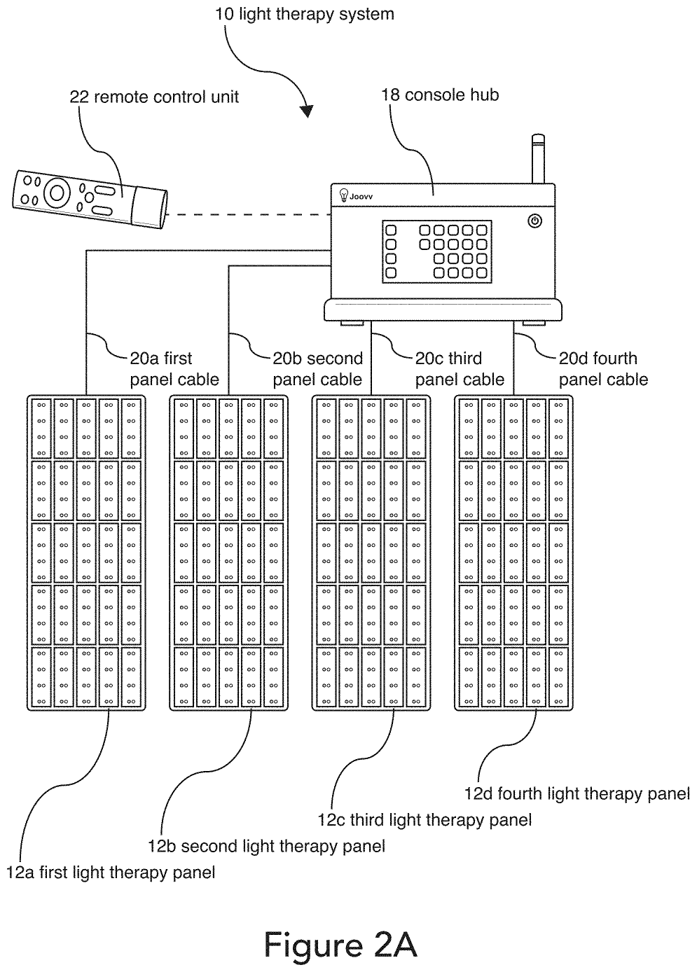

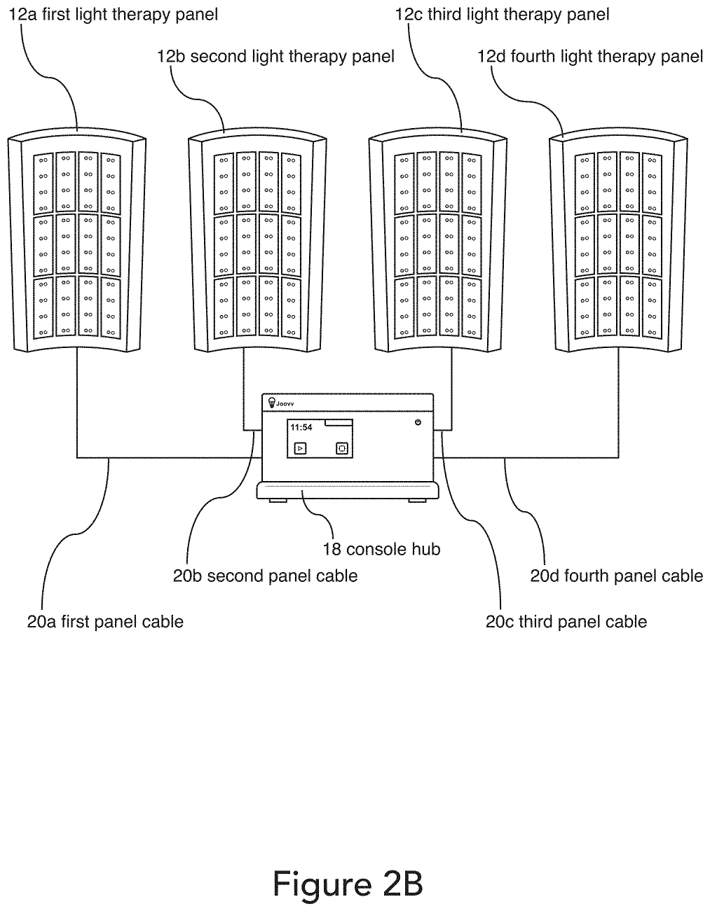

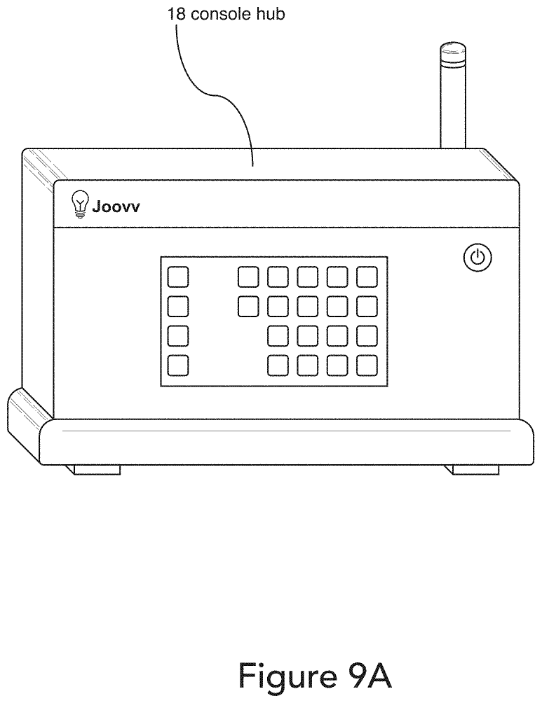

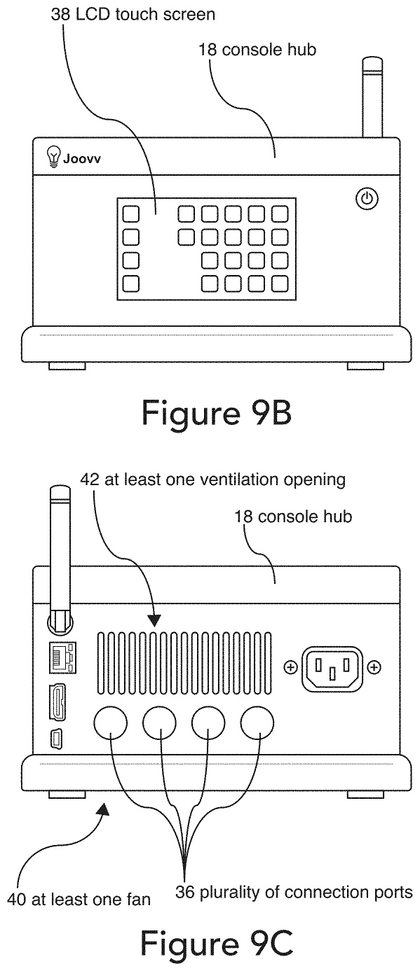

FIGS. 2A and 2B show the light therapy system 10 including a console hub 18, a remote control unit 22 (only shown in FIG. 2A), a first light therapy panel 12a, a second light therapy panel 12b, a third light therapy panel 12c, and a fourth light therapy panel 12d. FIGS. 2A and 2B also include the first panel cable 20a, second panel cable 20b, third panel cable 20c, and fourth panel cable 20d, which, in many embodiments, at least one of electrically and communicatively couple each light therapy panel 12 to the console hub 18. The console hub 18 may act as a central controller for each light therapy panel 12. The console hub 18 may be communicatively and electrically coupled to at least one light therapy panel 12 whereby the console hub 18 is configured to operate the at least one light therapy panel 12. Any communication protocol may be implemented by the remote control unit 22, console hub 18, and/or light therapy panel 12, such as WiFi, 3G, 4G, 5G, LTE, Bluetooth, Bluetooth low energy (“BLE”), Z-Wave, NFC, RFID, SigFox, DigiMesh, MiWi, Weightless, Thread, ZigBee, and the like. Further details of the console hub 18 will be discussed with reference to FIGS. 9A-9C.

The remote control unit 22 may be configured to operate at least one of the console hub 18 and the at least one light therapy panel 12, meaning the remote control unit 22 sends operation commands to the console hub 18 and/or the at least one light therapy panel 12. In some embodiments, the remote control unit 22 communicates via Bluetooth 4.1, which supports BLE. Furthermore, the remote control unit 22 may be battery powered, for example by two AAA alkaline batteries. The remote control unit 22 may further include an integrated digital microphone configured to receive voice commands.

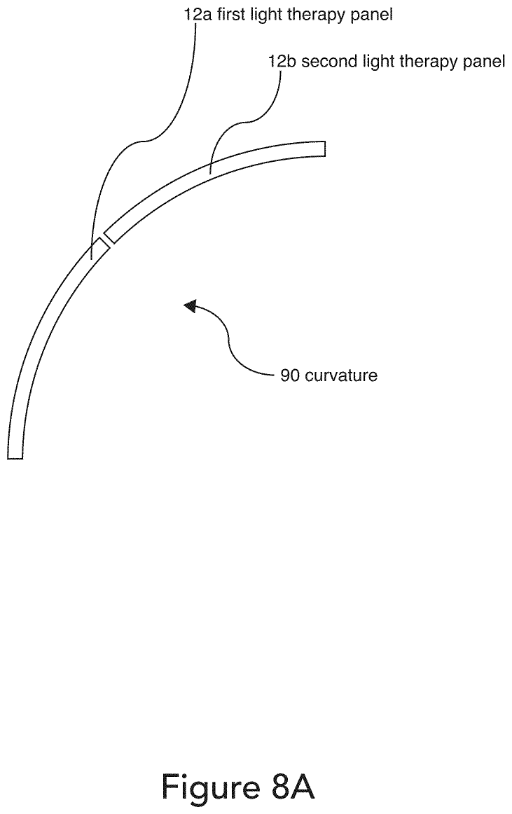

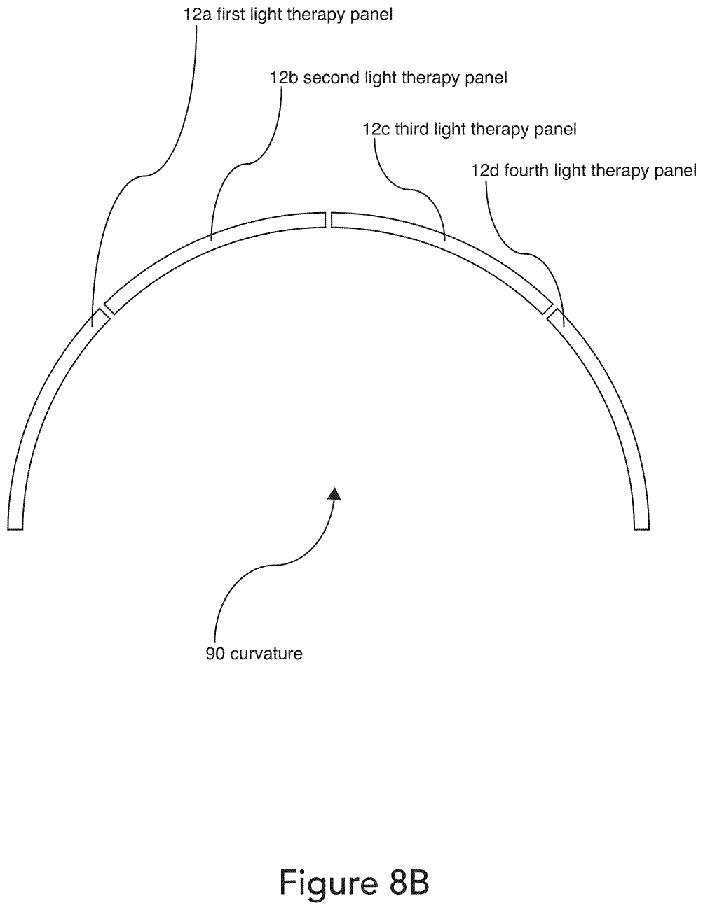

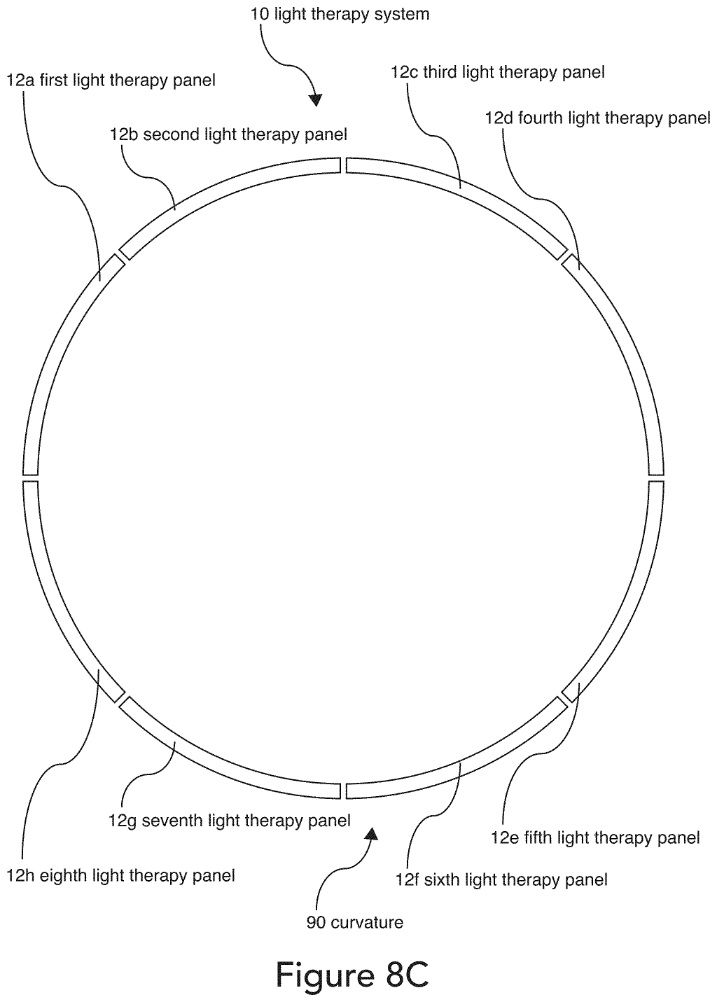

FIG. 2B specifically illustrates the light therapy panels 12a-d with curved housings. The light therapy panels 12a-d may have non-curved housings, as illustrated in FIG. 2A. In some embodiments, the curved housings are configured to at least partially surround a user 64 during light therapy treatment. Multiple users 64 at one time may use the light therapy system 10, where each user 64 uses a single light therapy panel 12. In some embodiments, the light therapy panels 12a-d are positioned such that they create a continuous curve, as shown in FIGS. 8A-8C. Multiple users 64 or a single user 64 may use the light therapy system 10 when the light therapy panels 12 are positioned in a continuous curve.

Each of the light therapy panels 12a-d shown in FIGS. 2A and 2B may include a housing 13 and a plurality of lights arranged and configured to emit at least one of red light and infrared light. The plurality of lights may also be configured to emit other types of light, as will be discussed with reference to FIGS. 6A-6C. In some embodiments, the plurality of lights are substantially evenly distributed across each light therapy panel 12a-d. The housing 13 of each of the light therapy panels 12a-12d may be a rigid housing 13. Each of the light therapy panels 12a-d may be at least one of communicatively and electrically coupled to the console hub 18.

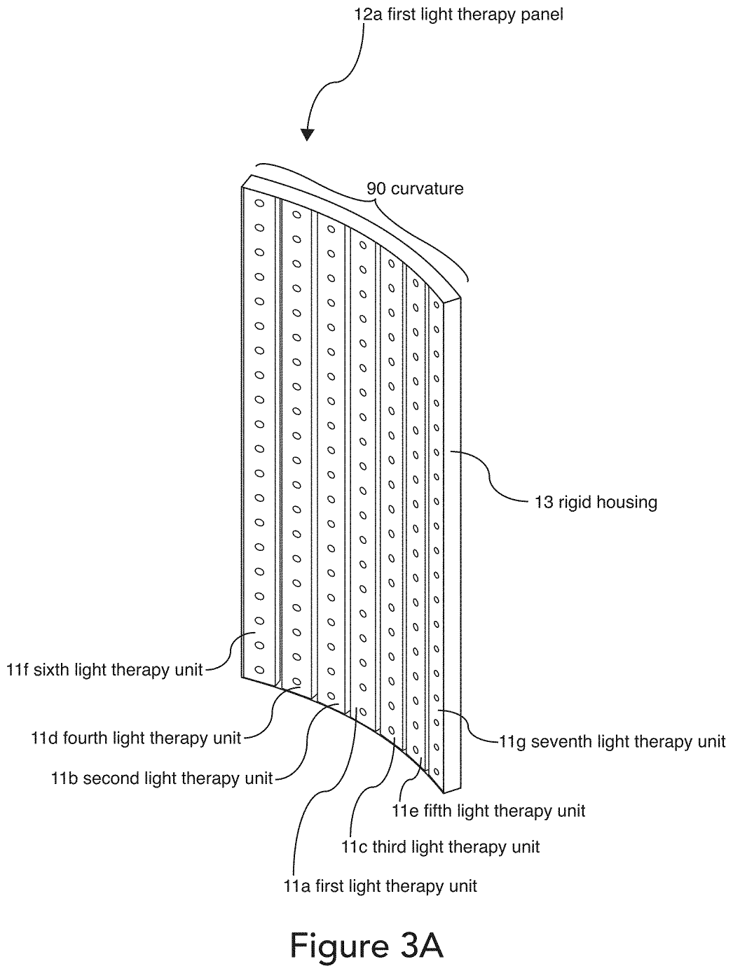

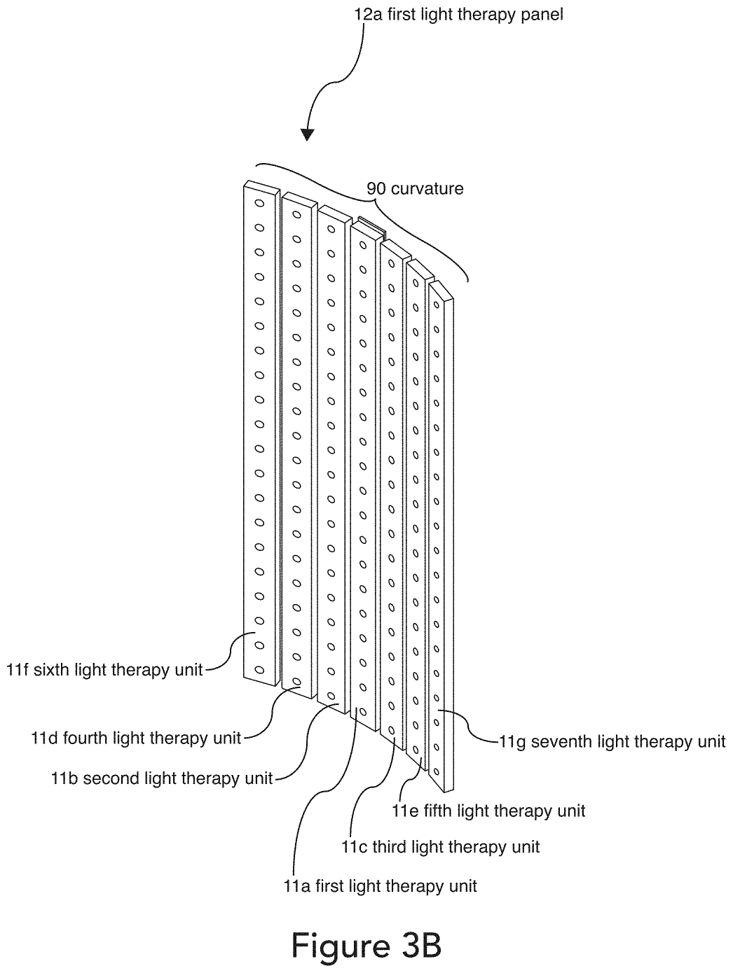

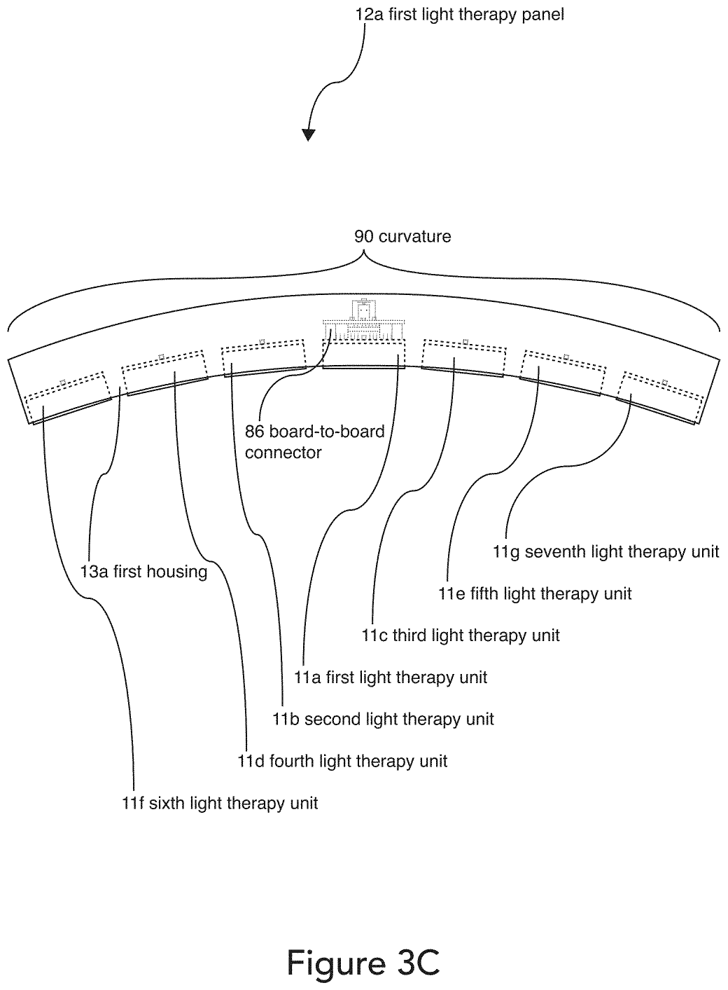

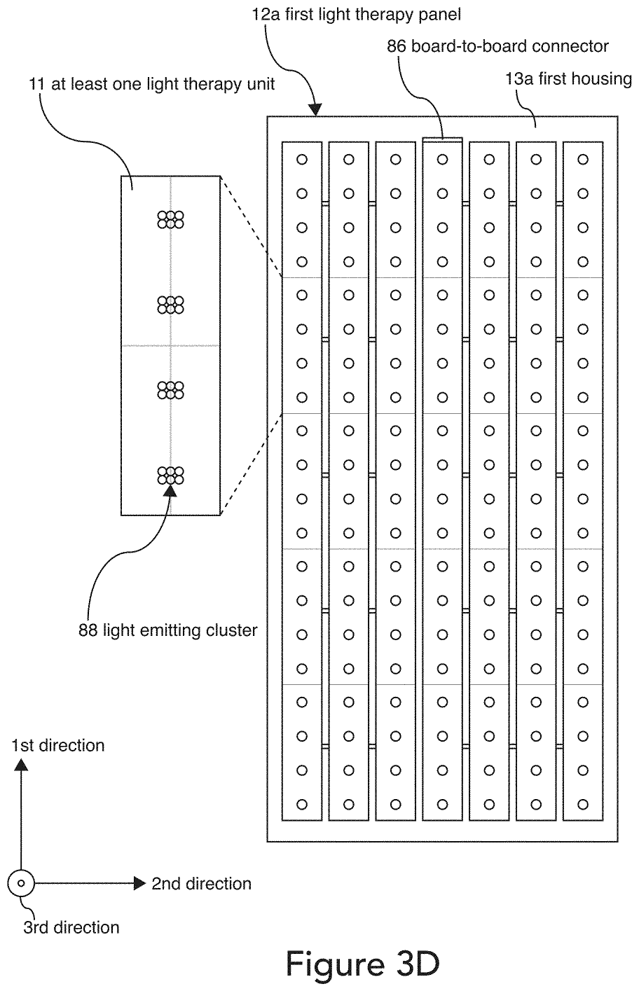

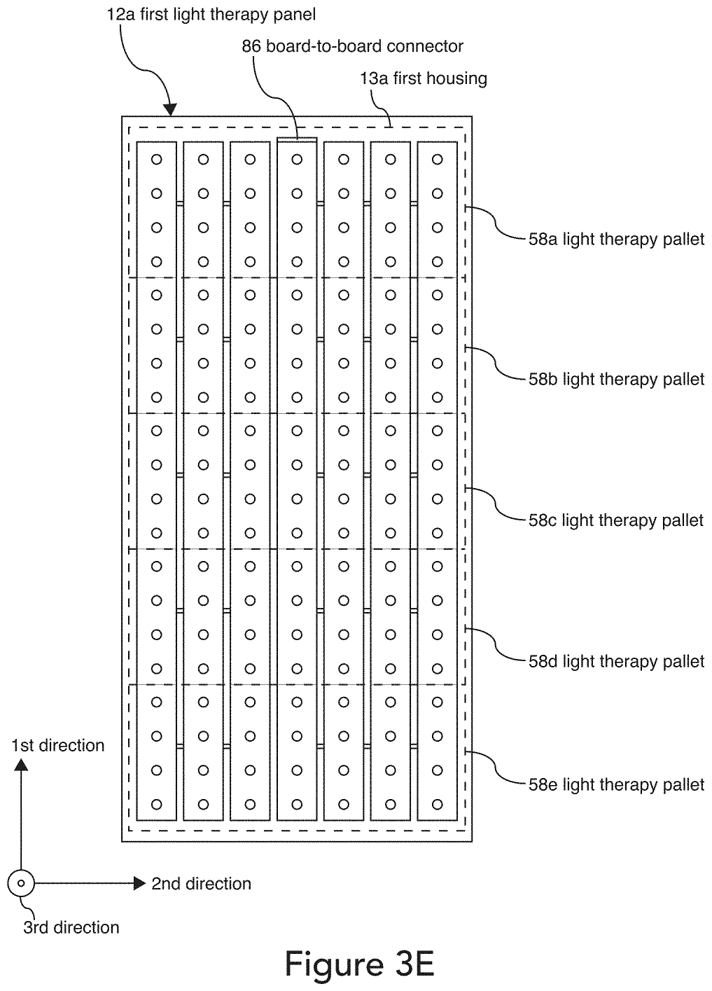

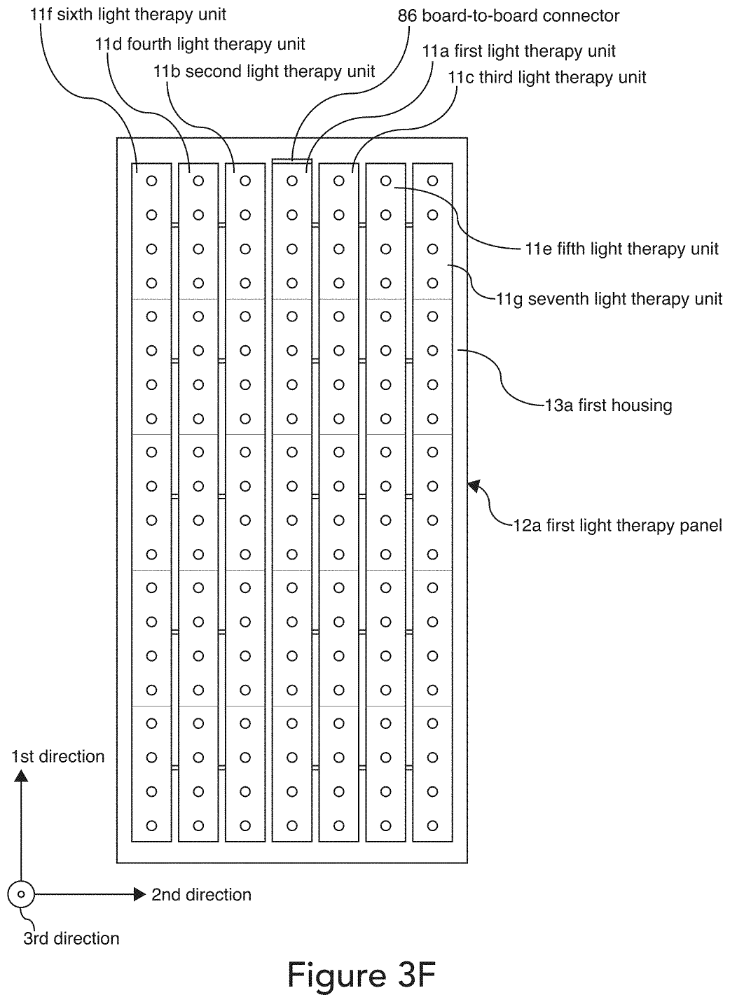

FIG. 3A shows a first light therapy panel 12a. FIG. 3B shows the same light therapy panel 12 from FIG. 3A, but FIG. 3B shows the light therapy panel 12 with the housing 13 removed, for clarity of illustration purposes. In some embodiments, the first light therapy panel 12a includes a first light therapy unit 11a, a second light therapy unit 11b, a third light therapy unit 11c, a fourth light therapy unit 11d, a fifth light therapy unit 11e, a sixth light therapy unit 11f, and a seventh light therapy unit 11g. As illustrated in FIG. 3A, the first light therapy unit 11a may be the center light therapy unit 11 in a row of light therapy units 11. The first light therapy unit 11a may be the light therapy unit 11 located on either end of the row, or may be located anywhere else in the row. In some embodiments, a row of the first light therapy panel 12a includes fewer than seven light therapy units 11. A row of the first light therapy panel 12a may include more than seven light therapy units 11.

FIG. 3C shows a top view of the first light therapy panel 12a, and better illustrates the curvature 90 of the panel 12a included in FIG. 3A. FIG. 3C also illustrates the first housing 13a which, in some embodiments, houses the at least one light therapy unit 11 of the first light therapy panel 12a. The first housing 13a may be a curved housing. In some embodiments, at least one of the light therapy units 11a-g at least partially protrudes through a front surface of the first housing 13a such that the treatment surface 32 of the first light therapy panel 12a is at least partially exposed. The first housing 13a may be configured such that the curvature of the housing 13 determines the curvature 90 of the first light therapy panel 12a. The curvature 90 may be defined by a radius, in some embodiments, the radius is about eighteen inches.

FIG. 3C also illustrates a board-to-board connector 86. In some embodiments, the board-to-board connector 86 is used to electrically, communicatively, and/or mechanically couple a first light therapy unit 11a to a second light therapy unit 11b, located above, below, and/or on either side of the first light therapy unit 11a. As such, a light therapy unit 11 may include a single board-to-board connector 86 or two board-to-board connectors 86; one located adjacent the top of the unit 11 and one located adjacent the bottom of the unit 11. The board-to-board connector 86 may also at least one of electrically and communicatively couple multiple light therapy units 11, by connecting at least one of the driver and controller boards of each light therapy unit 11. As shown in FIG. 3C, in some embodiments, only the first light therapy unit 11a includes a board-to-board connector 86. As such, there may be a plurality of board-to-board connectors 86 that extend up and down the center of a first light therapy panel 12a, connecting the first light therapy unit 11a in each row of light therapy units 11 to the next first light therapy unit 11a in the next row (above and/or below the prior row) of light therapy units 11. In some embodiments, each row of light therapy units 11 comprises a plurality of board-to-board connectors 86. For example, each row may include a board-to-board connector 86 on the first light therapy unit 11a, the fourth light therapy unit 11d, and the fifth light therapy unit 11 e. The board-to-board connector 86 may be located on any one or multiple light therapy units 11a-g in each row of light therapy units 11.

FIG. 3D shows a front view of the first light therapy panel 12a, including the first housing 13a, a board-to-board connector 86, and a zoomed-in view of at least one light therapy unit 11. As illustrated by the directional indicator in FIG. 3D, the at least one light therapy unit 11 is elongate along a first direction, which may be thought of as the direction generally described as “vertical” or “up and down”. The first light therapy panel 12a is shown with columns of five light therapy units 11 extending in the first direction. Each column of the first light therapy panel 12a may comprise more than five light therapy units 11 extending in the first direction. In some embodiments, the first light therapy panel 12a includes fewer than five light therapy units 11 per column extending in the first direction. While in some embodiments, the first light therapy panel 12a includes more than five light therapy units 11 per column extending in the first direction.

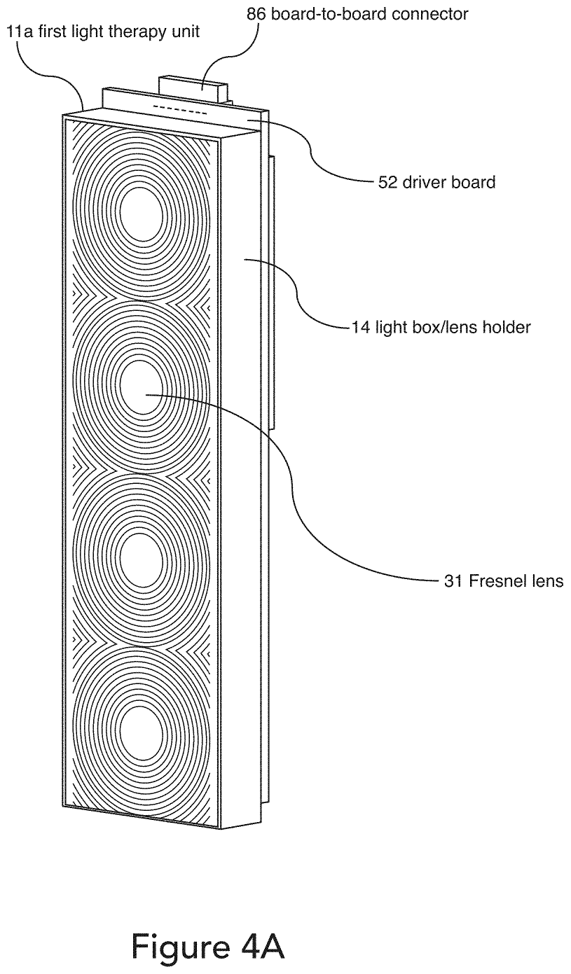

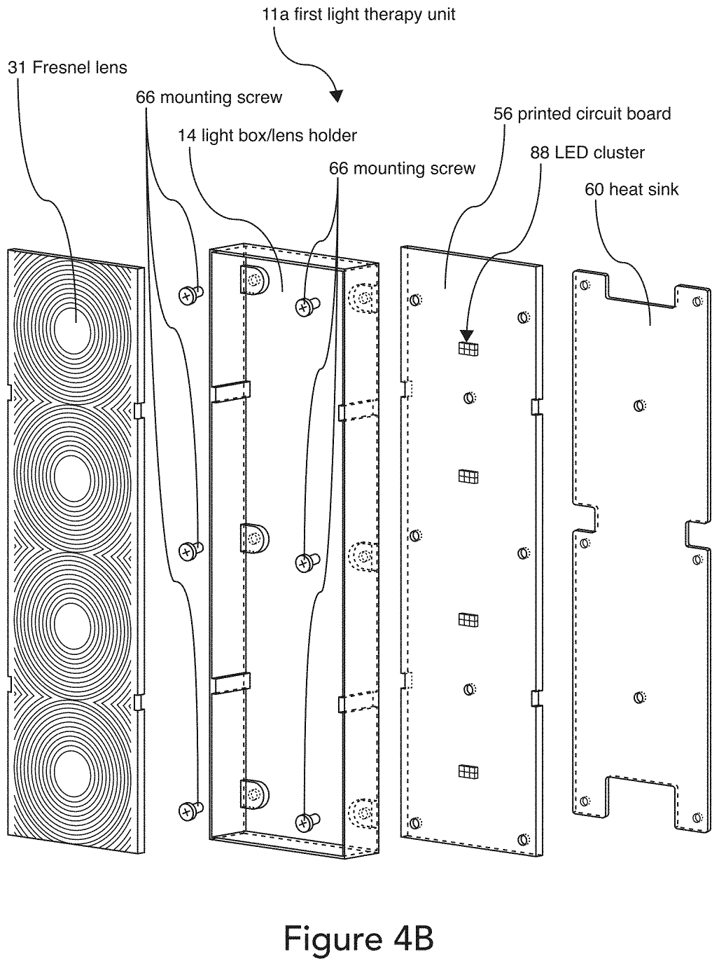

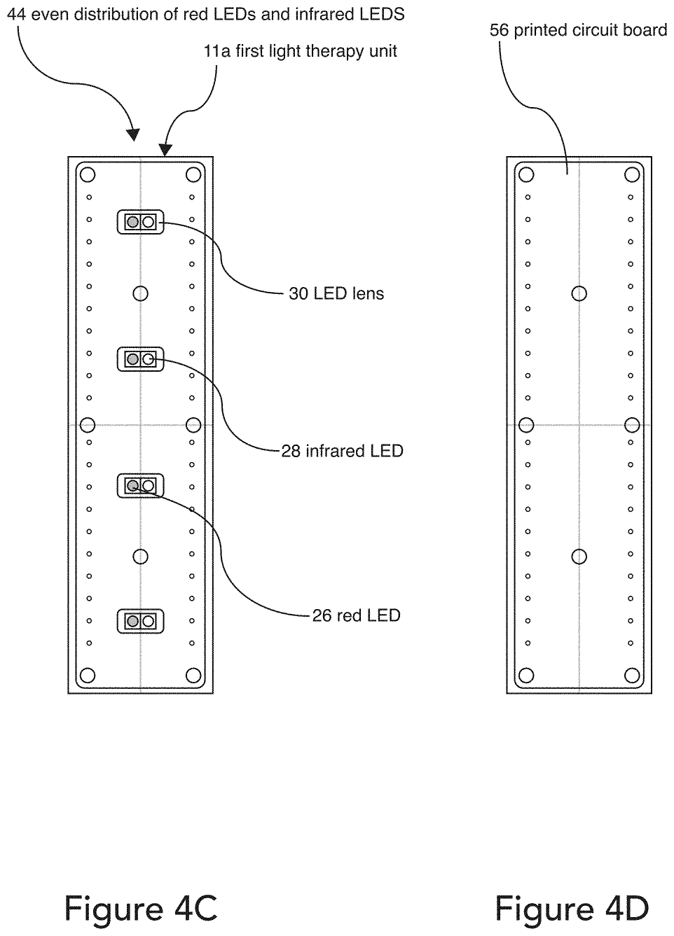

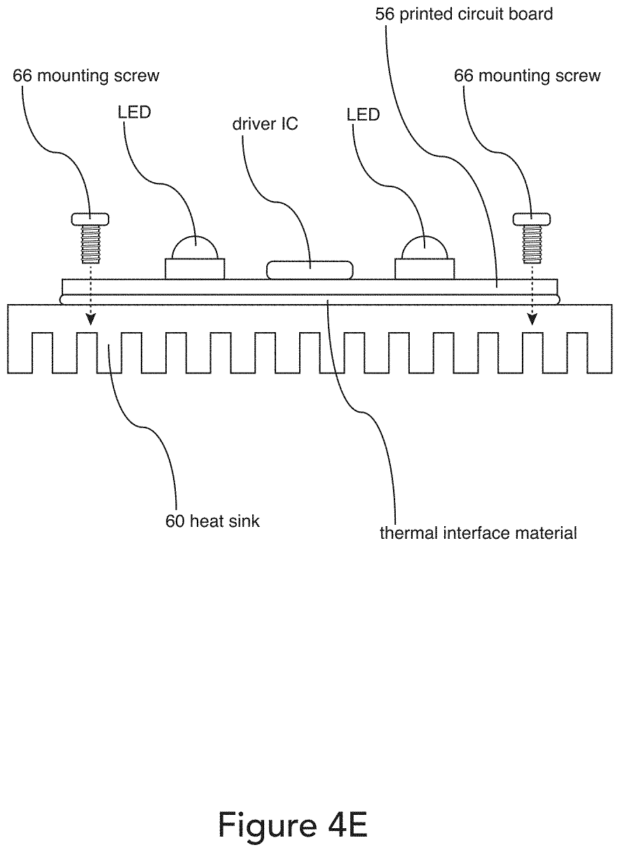

The enlarged view of the at least one light therapy unit 11 includes four LED clusters 88. In some embodiments, the at least one light therapy unit 11 includes more than four LED clusters 88, such as five, six, seven, or eight LED clusters 88. However, in some embodiments, the at least one light therapy unit 11 may include fewer than four LED clusters 88, such as one, two, or three LED clusters 88. The LED cluster 88 will be discussed in greater detail later in the disclosure. As indicated in FIG. 3D, as well as FIGS. 3E and 3F, the at least one light therapy unit 11 will be shown in FIG. 4A, and discussed further with reference thereto.

FIG. 3E is similar to FIG. 3D and again illustrates the first light therapy panel 12a, including the first housing 13a, board-to-board connector 86, and the enlarged view of the at least one light therapy unit 11. FIG. 3E also illustrates light therapy pallets 58a-e. It should be noted that the “row of light therapy units 11” previously discussed in the disclosure may be thought of as equivalent to a light therapy pallet 58, and the terms “row” and “pallet” may be used interchangeably in this context. As previously mentioned, each light therapy pallet 58 may comprise seven light therapy units 11a-g. Each light therapy pallet 58 may include more than seven light therapy units 11. In some embodiments, each light therapy pallet 58 includes fewer than seven light therapy units 11. As demonstrated in FIG. 3E, the first light therapy panel 12a may include five light therapy pallets 58a-e. In some embodiments, the first light therapy panel 12a comprises fewer than five light therapy pallets 58. The first light therapy panel 12a may include more than five light therapy pallets 58.

Similar to FIG. 3D, FIG. 3E includes a directional indicator. As previously mentioned, in many embodiments, the at least one light therapy unit 11 is elongate along the first direction. Columns of at least one light therapy unit 11 may extend along the first direction. As illustrated in FIG. 3E, in some embodiments, each light therapy pallet 58 extends along the second direction, which is perpendicular to the first direction. The second direction may be thought of as the direction generally described as “horizontal” or “side-by-side”. As each pallet 58 is comprised of at least one light therapy unit 11, the light therapy units 11 may also be thought of as aligned along the second direction. The plurality of light therapy pallets 58a-e may be aligned along the first direction with respect to one another. Stated differently, while each individual pallet 58 extends along the second direction, multiple pallets 58 are “stacked” on top of one another, and thus, are aligned along the first direction. The third direction, which in some embodiments is perpendicular to both the first direction and the second direction, may be used to describe the direction of the curvature of the first light therapy panel 12a. The third direction may also be thought of as the direction coming out of the page.

FIG. 3F is similar to FIGS. 3D and 3E and shows the first light therapy panel 12a with the first housing 13a, and board-to-board connector 86. In contrast to FIG. 3E, FIG. 3F labels individual light therapy units 11 on the first light therapy panel 12a, rather than each light therapy pallet 58. As shown in FIGS. 3B and 3C, in many embodiments, the first light therapy unit 11a is the center light therapy unit 11 in each pallet 58. The first light therapy unit 11a may be located between the second and third light therapy units 11b, 11c. In some embodiments, the second light therapy unit 11b is located adjacent the fourth light therapy unit 11d, which in turn is located adjacent the sixth light therapy unit 11f. The third light therapy unit 11c may be located adjacent the fifth light therapy unit 11e, which in turn is located adjacent the seventh light therapy unit 11g. In some embodiments, each light therapy unit 11a-g is at least one of mechanically, electrically, and communicatively coupled to at least one of the other light therapy units 11a-g. Methods of mechanical, electrical, and communicative coupling between light therapy units 11 will be discussed in more detail throughout the disclosure.

It should be noted that though the first light therapy panel 12a is expressly labeled in FIGS. 3A-3F, each of the second light therapy panel 12b, third light therapy panel 12c, and fourth light therapy panel 12d may be substantially identical to the first light therapy panel 12a, and thus, FIGS. 3A-3F may represent any and/or all of the first, second, third, and fourth light therapy panels 12a-d. This also applies to other Figures that show and label any single element (e.g., light therapy panel or light therapy unit) as the “first”. For example, though FIG. 4A is labeled as the first light therapy unit 11a, it may also represent any one of the second, third, fourth, fifth, sixth, and/or seventh light therapy units 11b-g.

FIG. 4A illustrates a front perspective view of a first light therapy unit 11a. In many embodiments, each light therapy unit of the plurality of light therapy units 11 includes a light box/lens holder 14 and a Fresnel lens 31. As will be discussed with reference to FIG. 4C, the first light therapy unit 11a may include an LED lens 30. In some embodiments, the LED lens 30 is not a Fresnel lens 31. The LED lens 30 may be a Fresnel lens 31, as demonstrated in FIG. 4A. In some embodiments the light box/lens holder 14 is rigid. The light box/lens holder 14 may be non-rigid. The lens, whether a Fresnel lens 31 or another LED lens 30, may be incorporated into the light box/lens holder 14, such that the lens is integral to the light box/lens holder 14. As shown, a first light therapy unit 11a may also include a driver board 52 and at least one board-to-board connector 86. In some embodiments, any and/or all of the light therapy units in the plurality of light therapy units 11 include a driver board 52 and at least one board-to-board connector 86. In some embodiments, any and/or all of the light therapy units in the plurality of light therapy units 11 include either the driver board 52 or the board-to-board connector 86. In some embodiments, the measurements of a single light therapy unit 11 are about 180 mm tall by 50 mm wide. A single light therapy unit 11 may measure larger or smaller than the listed dimensions. FIG. 4B shows an exploded view of the first light therapy unit 11a shown in FIG. 4A. As previously mentioned, the first light therapy unit 11a may include a Fresnel lens 31 and a light box/lens holder 14. In some embodiments, the Fresnel lens 31 comprises a solid pane mounted on the light box/lens holder 14. The Fresnel lens 31 may clip onto the light box/lens holder 14. In some embodiments, the Fresnel lens 31 comprises an acrylic panel. The Fresnel lens 31 may comprise individual lenses rather than a solid pane including each lens. As shown in FIG. 4B, the first light therapy unit 11a may further include a printed circuit board 56. In some embodiments, the printed circuit board 56 includes at least one LED cluster 88. Each LED cluster 88 may include six light emitting sources, such as LEDs or other light sources. Each LED cluster 88 may include fewer than six light emitting sources, such as five, four, three, two, or one light emitting source. In some embodiments, each LED cluster 88 includes more than six light emitting sources, such as seven, eight, ten, twelve, or more light emitting sources. As illustrated in FIG. 4B, the printed circuit board 56 may include four LED clusters 88. The four LED clusters 88 may be aligned along the first direction, such that the four LED clusters 88 align along the same direction of extension of the first light therapy unit 11a. In some embodiments, the printed circuit board 56 includes more than four LED clusters 88. The printed circuit board 56 may include fewer than four LED clusters 88.

In some embodiments, and as demonstrated in FIG. 4B, the first light therapy unit 11a comprises four LED clusters 88 in line with a single-pane Fresnel lens 31 including four lenses. Each lens in the Fresnel lens 31 may be configured to line up with each LED cluster 88 such that the light emitted by each LED cluster 88 is focused through each lens in the Fresnel lens 31. At least one of the Fresnel lenses 31 and the plurality of LED clusters 88 may comprise more or fewer than four lenses and four LED clusters 88. The first light therapy unit 11a may be configured such that the number of LED clusters 88 substantially always matches the number of individual lenses on the Fresnel lens 31. The Fresnel lens 31 may act as both a reflector and a lens. The Fresnel lens 31 may not include a reflector. The Fresnel lens 31 may be configured to produce continuous, uniform light output as a result of the even distribution of LED clusters 88 across the panel surface of the Fresnel lens 31. By virtue of the acrylic panel design, the Fresnel lens 31 may also have less “dead space” than other types of LED lenses 30.

In some embodiments, the first light therapy unit 11a also includes a heat sink 60. In some embodiments, the heat sink 60 is mechanically coupled to at least one of the Fresnel lens 31, the light box/lens holder 14, and the printed circuit board 56 via a plurality of mounting screws 66, friction fit, and the like. Any suitable coupling mechanism may be used to couple the heat sink 60 to the first light therapy unit 11a. In many embodiments, the heat sink 60 is configured to enable heat regulation of the first light therapy unit 11a by dissipating the heat produced by the printed circuit board 56, LED clusters 88, and any other component producing heat, to the external environment via convection cooling. The heat may be transferred from the heat producing components by the flow of fluid surrounding the heat sink. In some embodiments, the fluid is air. In some embodiments, the fluid is any other type of suitable fluid or liquid.

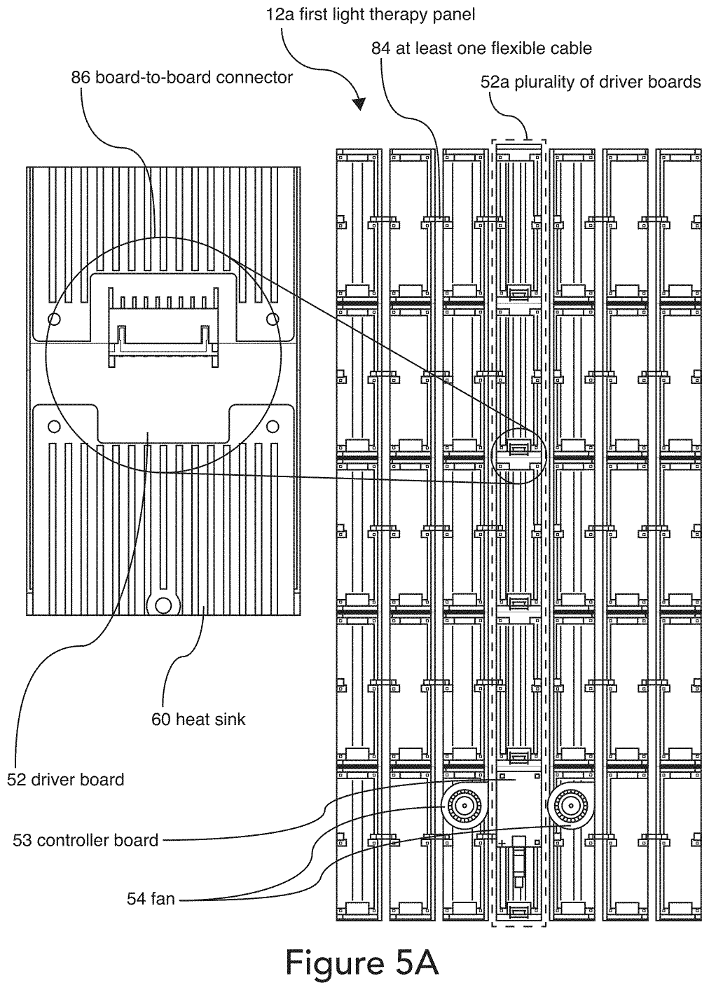

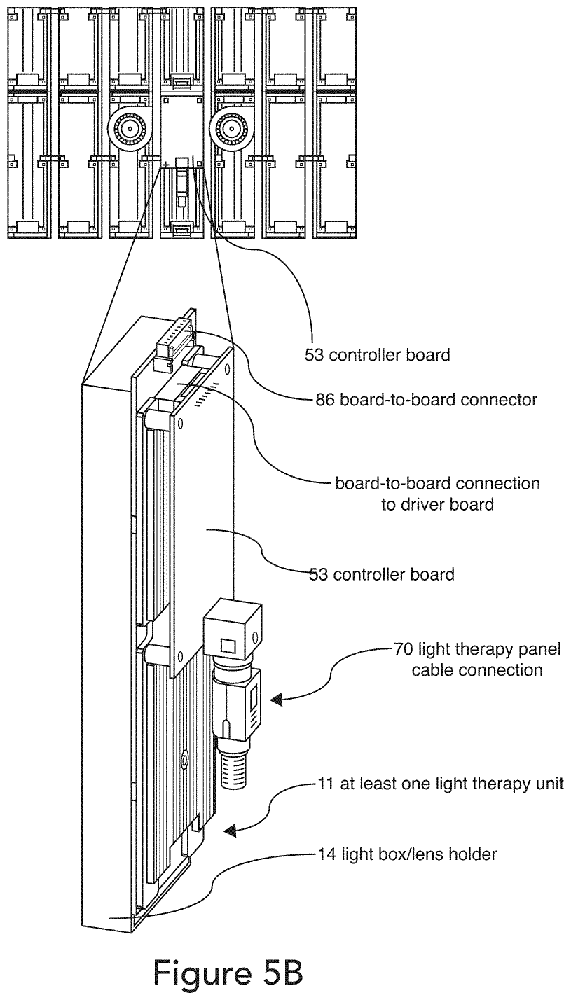

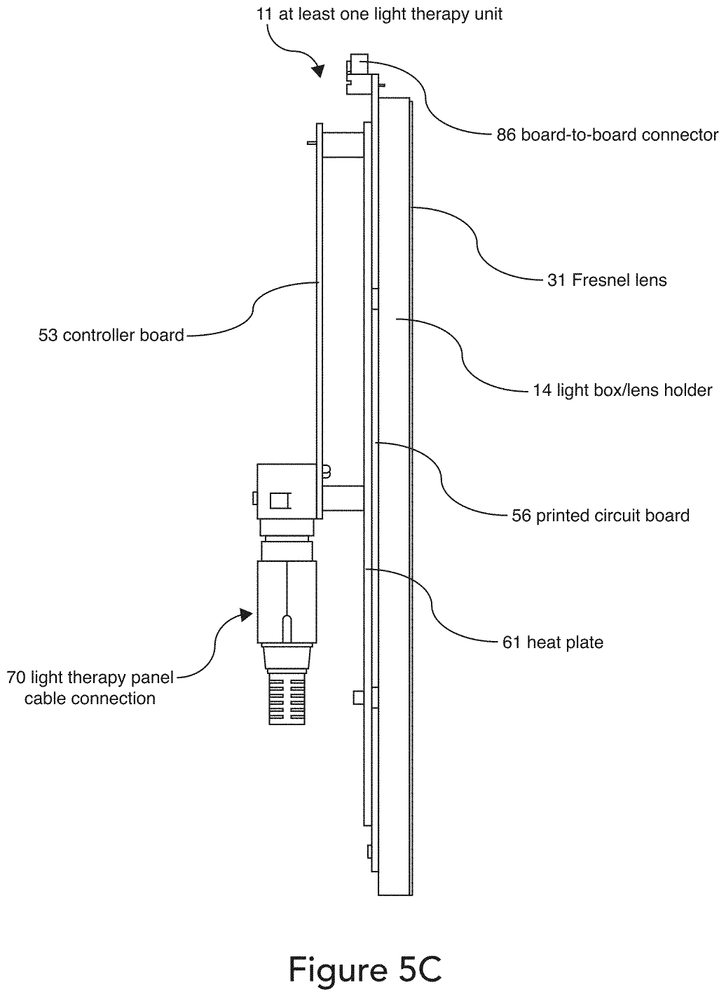

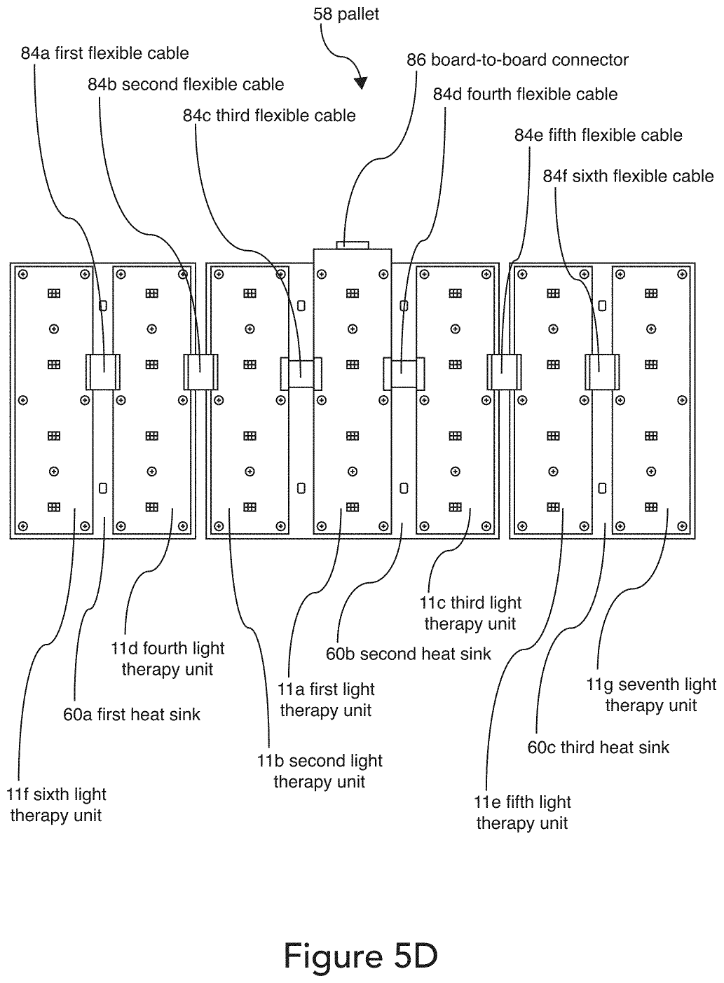

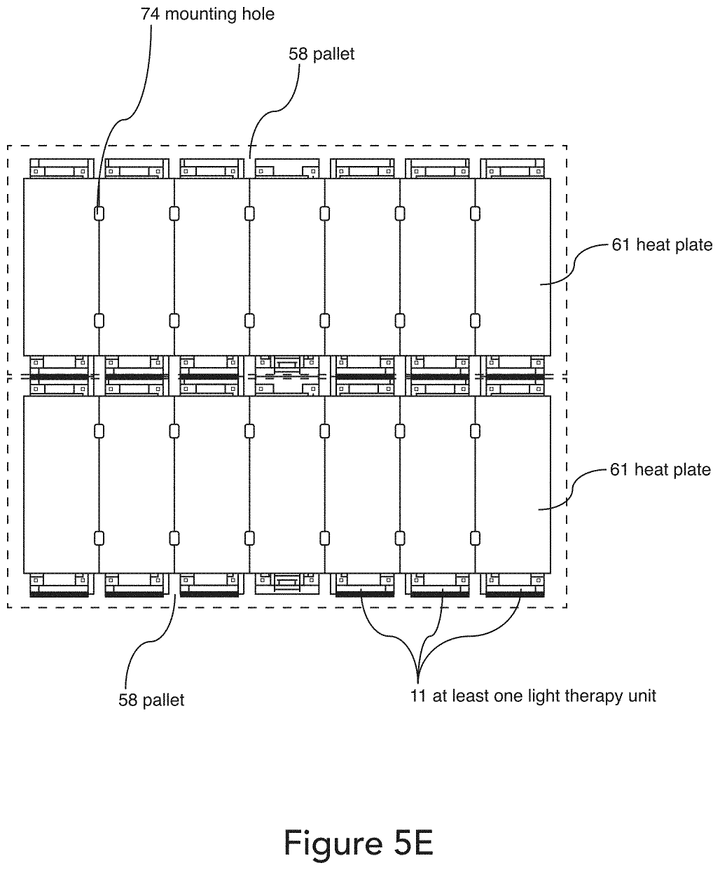

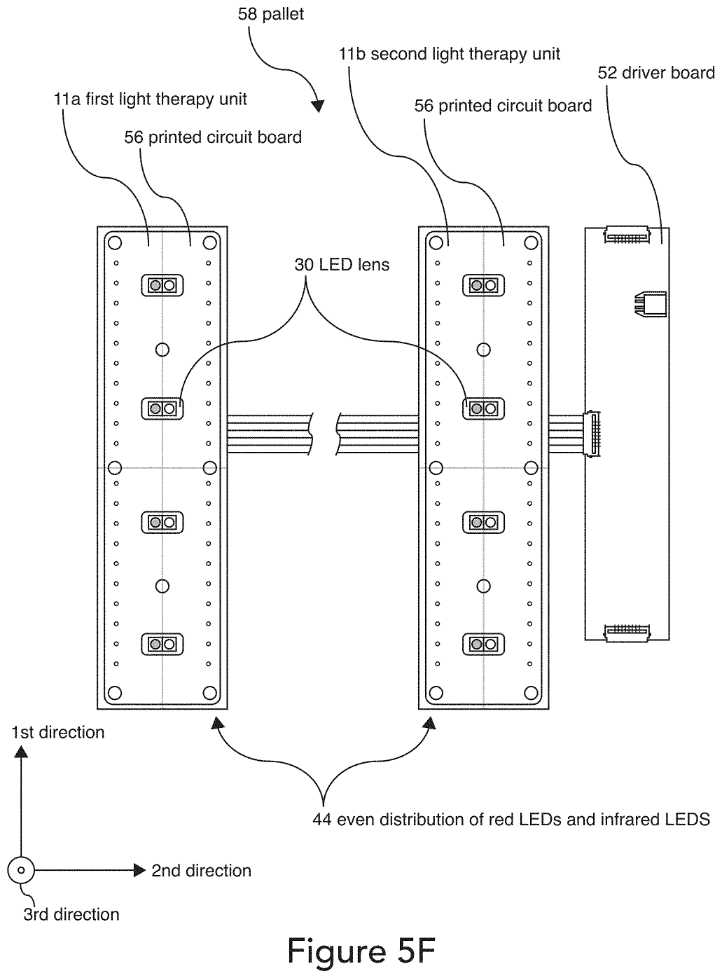

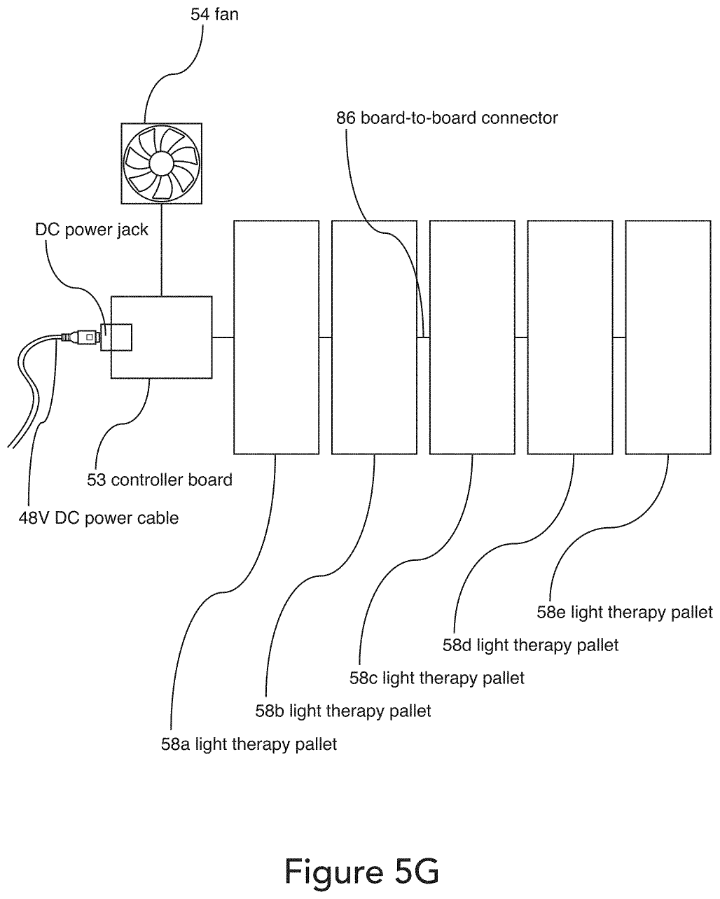

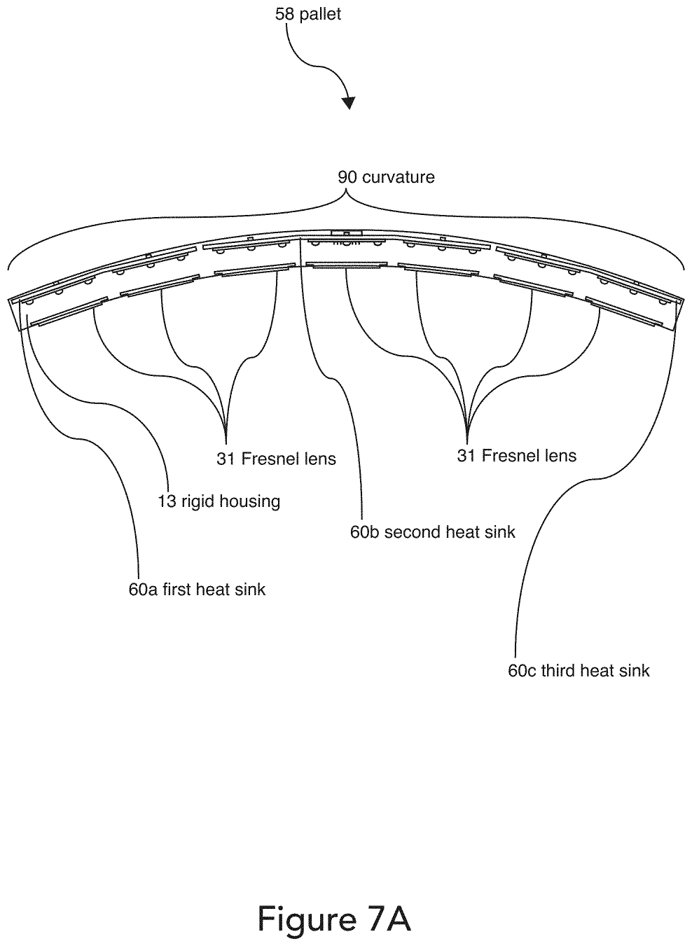

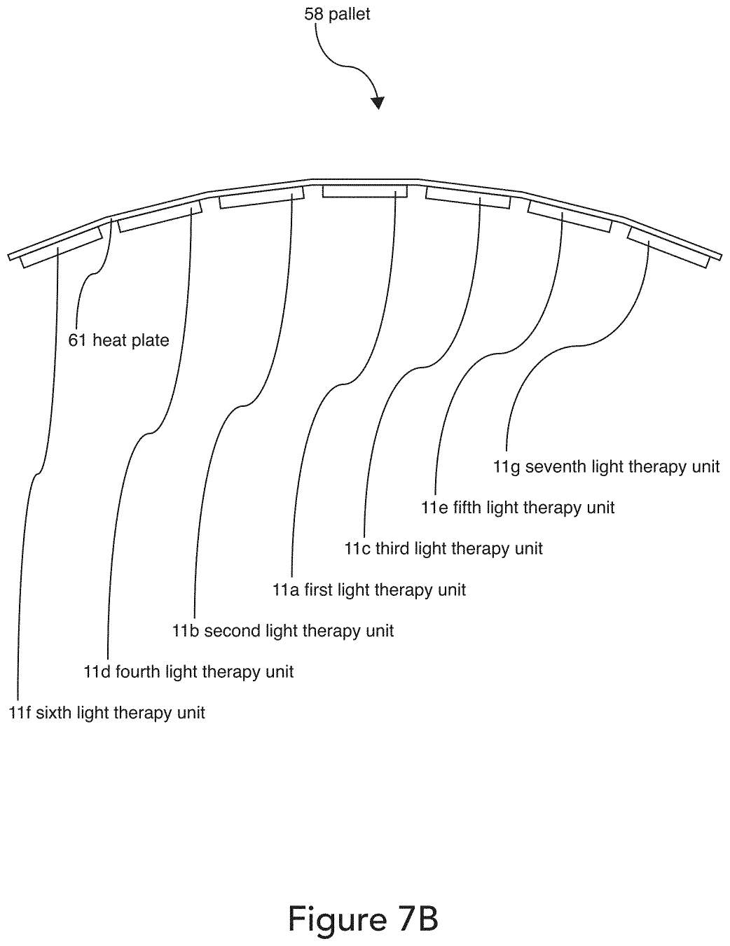

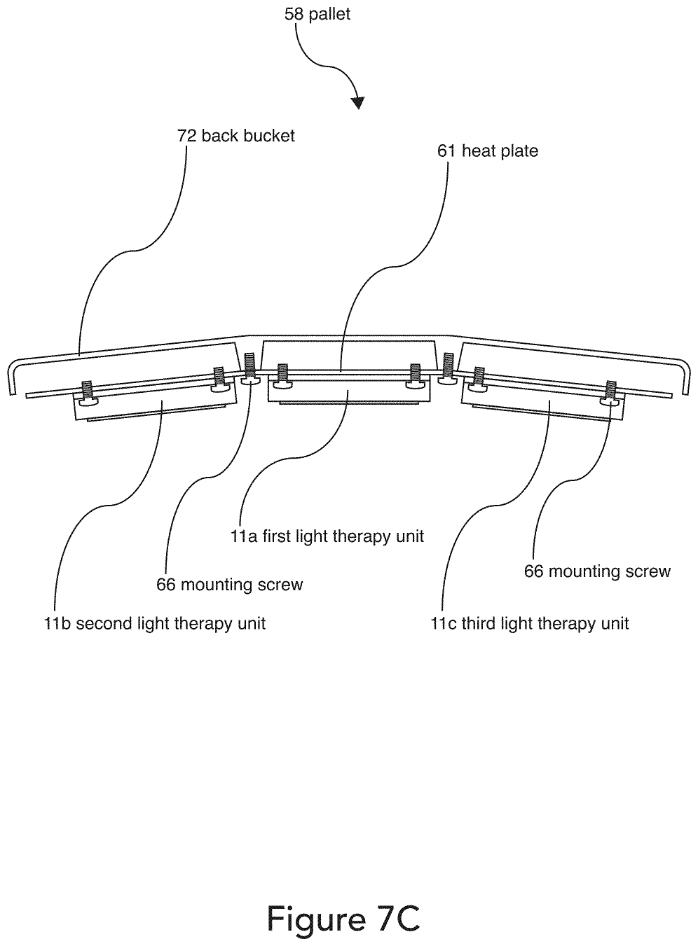

The heat sink 60 may be comprised of aluminum or an aluminum alloy. In some embodiments, the heat sink 60 is comprised of another material, such as magnesium, in replacement of or in addition to aluminum and/or an aluminum alloy. The heat sink 60 may be comprised of any material with a suitable level of thermal conductivity. In some embodiments, the heat sink 60 is sized and configured to fit a single light therapy unit 11. The heat sink 60 may be sized and configured to fit multiple light therapy units 11, as will be discussed later in the disclosure, in particular with reference to FIGS. 5D, 5E, and 7A-7C.

FIGS. 4C and 4D illustrate another embodiment of a first light therapy unit 11a. The most notable differences between the embodiment portrayed in FIGS. 4C and 4D and the embodiment portrayed in FIGS. 4A and 4B are the LED lens 30 and the presence of only two LEDs per cluster: a red LED 26 and an infrared LED 28. FIGS. 4C and 4D illustrate front and back views, respectively, of a printed circuit board 56 of a first light therapy unit 11a, according to some embodiments. As shown by FIG. 4C, each printed circuit board 56 may comprise eight LEDs mounted and heatsinked on the printed circuit board 56. In many embodiments, four LEDs are configured to emit red light and four LEDs are configured to emit infrared light. Further, two of the red LEDs 26 may emit light of a wavelength of 630 nm, and the other two red LEDs 26 may emit light of a wavelength of 660 nm. In many embodiments, IR light is emitted of a wavelength of 850 nm. Each pair of LEDs—one red 26 and one infrared 28—may share a single lens 30 such that it appears to a user as though only one light is shown through each lens 30. In some embodiments, each printed circuit board 56 is an FR4 printed circuit board with exposed copper configured to connect to the top printed circuit board 56 and the thermal pads of each LED.

It should be noted that while the specification often refers to red and infrared light, any type of light may be implemented with the system 10, such as blue light, green light, near-infrared light, and/or any forms of light on the visible spectrum and infrared spectrum. In many embodiments, the system 10 can operate and emit light of a wavelength between about 400 nm and 1200 nm. The spectrum of light emission of the system 10 will be discussed further with reference to FIGS. 6A-6F.

FIG. 4E shows a side view of certain components of a light therapy unit 11, including a heat sink 60, a printed circuit board 56, a thermal interface material, a driver IC, mounting screws 66 and multiple LEDs. In some embodiments, the thermal interface material is located between the printed circuit board 56 and the heat sink 60 to ensure sufficient pressure and interface area between the printed circuit board 56 and the heat sink 60. As illustrated in FIG. 4E, the LEDs and the driver IC may be mounted on the printed circuit board 56. The light therapy unit 11 may include the mounting screws 66 to mount the printed circuit board 56 to the heat sink 60. In some embodiments, as shown in FIG. 4E, two mounting screws 66 are used. Any number of mounting screws 66, or other attaching mechanisms, may be used to couple the printed circuit board 56 to the heat sink 60.

FIG. 5A illustrates a back view of a first light therapy panel 12a, and includes an enlarged view of a board-to-board connector 86. In some embodiments, the board-to-board connector 86 is configured to at least one of mechanically, electrically, and communicatively couple a single light therapy unit 11 to another light therapy unit 11. As previously stated, the center light therapy unit 11 in each pallet 58 may include a board-to-board connector 86. In some embodiments, the board-to-board connector 86 is configured to couple at least one of the driver board 52 and controller board 53 of a single light therapy unit 11 to at least one of the driver board 52 and controller board 53 of another light therapy unit 11. The first light therapy panel 12a may include a plurality of driver boards 52a extending along the first direction up the first light therapy panel 12a.

FIG. 5A also includes at least one flexible cable 84. In many embodiments, the at least one flexible cable 84 is configured to mechanically couple each light therapy unit 11 of the at least one light therapy unit 11 to its adjacent light therapy unit along the second direction. As previously discussed in the disclosure, each light therapy panel 12 of the system 10 may define a curved shape. In some embodiments, the curvature is achieved by the at least one flexible cable 84 allowing the light therapy units 11 in each pallet 58 to move with respect to one another along the third direction. For example, in order to achieve a substantially uniform curvature, the first light therapy unit 11a may be located the furthest back along the third direction while the sixth and seventh light therapy units, 11f, 11g (i.e., the end units) are located the furthest forward along the third direction. The second, third, fourth, and fifth light therapy units 11b-e may be located evenly along the curve between the center (i.e., first) light therapy unit 11a and the end (i.e., sixth or seventh) light therapy units 11f or 11g. In some embodiments, the at least one flexible cable 84 is also configured to extend along the second direction such that the distance between light therapy units 11 extends along the second direction. Stated differently, the at least one flexible cable 84 may enable widening of the light therapy panel 12 by extending the distance between individual light therapy units 11 in a pallet 58. As such, the at least one flexible cable 84 facilitates an adjustable curvature 90 of each light therapy panel 12.

FIG. 5A also illustrates a fan 54 located on either side of the controller board 53. In some embodiments, the fan 54 is a side blow fan. The fan 54 may be any type of fan suitable to assist with cooling the first light therapy panel 12a, including a traditional style fan. Though not shown in FIG. 5A with a housing 13, the fan(s) 54 may be located inside the housing 13 of a light therapy panel 12. In some embodiments, the fan(s) 54 is located at least partially inside the housing 13. The housing 13 may also include at least one ventilation opening. In some embodiments, the housing 13 does not include a fan 54 and/or a ventilation opening.

FIG. 5B shows another back view of a light therapy panel 12, and includes an enlarged side and back view of a light therapy unit 11. In some embodiments, the light therapy unit 11 includes a controller board 53, a board-to-board connector 86, a board-to-board connection to driver board, and a light therapy panel cable connection 70, all coupled to a light box/lens holder 14. The controller board 53 may be at least one of electrically and communicatively coupled to the console hub 18 via a panel cable 20 (shown in FIGS. 2A and 2B). The panel cable 20 may couple to the controller board 53 via the light therapy panel cable connection 70. The at least one of electrical and communicative coupling with the console hub 18 may enable the controller board 53 to send to and/or receive from the console hub 18 data, commands, and the like; thereby enabling the controller board 53 to control the light therapy unit 11.

In addition, the controller board 53 may be at least one of electrically and communicatively coupled to a controller board 53 of another light therapy unit 11 via at least one of the board-to-board connector 86 and the board-to-board connection to driver board. The board-to-board connector 86 and the board-to-board connection to driver board may represent the same element of the light therapy system 10. In some embodiments, the two elements are different, but similar. In many embodiments, the board-to-board connector 86 and/or the board-to-board connection to driver board enables the connection between the controller board 53 and the driver board 52 without the use of cables. Board-to-board connection between light therapy units 11 of a light therapy panel 12 may help facilitate light therapy treatment by organizing the light therapy units 11 and ensuring that light is emitted from each unit 11 in the proper sequence, for the proper amount of time, at the proper wavelength, and the like. Board-to-board communication may also enable substantially synchronized light emission from each light therapy unit 11 across a light therapy panel 12, such that there is a substantially continuous light output from the panel 12.

FIG. 5C illustrates a side view of a light therapy unit 11, similar to the enlarged view shown in FIG. 5B. As shown, the light therapy unit 11 may include a light box/lens holder 14, a printed circuit board 56, a board-to-board connector 86, a controller board 53, a light therapy panel cable connection 70, and a heat plate 61. The heat plate 61 may be very similar to the previously discussed heat sink 60, and will be discussed in further detail later in the disclosure. As mentioned with reference to FIG. 5B, the light therapy panel cable connection 70 may be used to couple a panel cable 20 to the light therapy unit 11 to thereby at least one of mechanically, electrically, and communicatively couple the controller board 53 to the console hub 18. In some embodiments, the light therapy panel cable connection 70 is configured to receive any kind of cable that will carry sufficient power to the light therapy unit 11.

The light therapy panel 12 may require 48V of power to operate, and, in some embodiments, the 48V are distributed across the light therapy panel 12. The 48V may be distributed across five pallets 58a-e, with one connection per pallet 58 (at the light therapy panel cable connection 70 on the first light therapy unit 11a). Reducing the number of cable connections by having one per pallet 58 rather than one per light therapy unit 11 may enable the system 10 to run more efficiently and at a higher voltage than would be possible with a greater number of connections and the resultant current drops at each connection. In some embodiments, the system 10 is configured to use IDT technology.

FIG. 5D illustrates a front view of a pallet 58 including seven light therapy units 11a-g and three heat sinks 60a-c. In addition, FIG. 5D includes six flexible cables 84a-f, which, in many embodiments, mechanically couple each light therapy unit 11 to its adjacent light therapy unit 11. Also, as previously discussed, the flexible cables 84a-f may be used to create the curvature 90 of each pallet 58, and thereby, the curvature 90 of a light therapy panel 12. It should be noted that though the first light therapy unit 11a is illustrated as slightly taller than the second, third, fourth, fifth, sixth, and seventh light therapy units 11b-g, in many embodiments, the first light box/lens holder 14a of the first light therapy unit 11a is substantially the same height as the light boxes/lens holders 14 of the other light therapy units 11b-g. The board-to-board connector 86 may cause the first light therapy unit 11a to be slightly taller than light therapy units 11 without the board-to-board connector 86.

In some embodiments, as shown in FIG. 5D, the pallet 58 includes a first heat sink 60a, a second heat sink 60b, and a third heat sink 60c. The first heat sink 60 may be mechanically coupled to the first light therapy unit 11a, the second light therapy unit 11b, and the third light therapy unit 11c. The second heat sink 60b may be mechanically coupled to the fourth light therapy unit 11d and the sixth light therapy unit 11f. The third heat sink 60c may be mechanically coupled to the fifth light therapy unit 11e and the seventh light therapy unit 11g. In some embodiments, each heat sink 60a-c is at least partially located within a housing 13 of the light therapy panel 12. The heat sinks 60a-c may be coupled to the light therapy units 11a-g via a plurality of screws or a similar mounting mechanism.

As shown in FIG. 5D, a heat sink 60 may be configured to couple to a plurality of light therapy units 11, such as two or three units 11. FIG. 5E illustrates that, in some embodiments, all seven light therapy units 11 are coupled to the same heat plate 61. It should be noted that in terms of materials and function, the heat sink 60 and heat plate 61 of this disclosure may be substantially the same. The heat plate 61 may differ from the heat sink 60 based upon size and the number of light therapy units 11 coupled to the heat plate 61. In some embodiments, the heat plate 61 is comprised of a blend of aluminum alloy. The mounting holes 74 included in FIG. 5E may be configured to enable mounting, via mounting screws 66, of the heat plate 61 on a back bucket 72, which will be discussed with reference to FIG. 7C. Similar to the heat sink 60, the heat plate 61 may be mechanically coupled to the light therapy units 11 via a plurality of mounting screws 66 or other similar attaching mechanism. In some embodiments, FIG. 5E shows two heat plates 61; one per pallet 58.

FIG. 5F illustrates a perspective view of another embodiment of the system 10, including an external driver board 52 at least one of electrically and communicatively coupled to a pallet 58, according to some embodiments. Though only two light therapy units 11a-b are shown in the figure, it should be understood that five light therapy units are implied, thus creating a pallet 58. In some embodiments, a driver board 52 with a 6-PIN FPC connection is electrically coupled to the pallet 58. The driver board 52 may have an input power of 48 Volts. The driver board 52 may also have multiple connection areas to facilitate the electrical coupling of one driver board 52 to multiple pallets 58, thus enabling a single driver board 52 to power and control an entire light panel 12. It should be noted that FIG. 5F also displays the embodiments shown in FIGS. 4C and 4D, comprising two LEDs per cluster and individual LED lenses 30. As shown, in some embodiments, the system 10 includes an even distribution of red LEDs and infrared LEDs 44 across each light therapy unit 11. In an embodiment with a greater variety of LEDs, the system 10 may still include an even distribution of LEDs.

FIG. 5G shows a schematic view of at least some of the electrical components of the system 10, including a 48V DC power cable, a DC power Jack, a controller board 53, a fan 54, and five light therapy pallets 58a-e. The schematic also includes the board-to-board connectors 86 extending across roughly the middle of each pallet 58 and the controller board 53. As previously stated, the board-to-board connectors 86 may be configured to connect the center, or first, light therapy unit 11a in each pallet 58 to the first light therapy unit 11 a in the next pallet 58. In some embodiments, the system 10 includes at least one fan 54. The fan 54 may be a DC fan or any other suitable type of fan. In some embodiments, the system 10 does not include a fan 54. As illustrated in FIG. 5G, the DC power jack may be integrated into the controller board 53 such that when the 48V DC power cable plugs in to the DC power jack, it is plugged in to the controller board 53. The system 10 may include additional electrical elements not included in this schematic. In some embodiments, one or more of the elements represented in FIG. 5G is not included in the system 10.

FIG. 6A illustrates a graph showing relative spectral power distribution vs. wavelength (in nm) for six different colors of light, including: royal blue, blue, green, amber, red, and near infrared. The graph specifically shows at what wavelength each light reaches maximum intensity, or 1.0 on the intensity scale. In some embodiments, the wavelengths are thus: royal blue at 445 nm, blue at 460 nm, green at 525 nm, amber at 595 nm, red at 650 nm, and NIR at 850 nm. These values may be approximate, such that the true maximum intensity of blue light may be reached at a wavelength between 450 and 470 nm, not necessarily exactly 460 nm. In some embodiments, each color of light is capable of being emitted across a much wider wavelength spectrum, but at lower intensity than at the peak wavelength. The spectrums of emission for each color of light, as shown in the graph are (in approximate values): 380 nm to 505 nm for royal blue, 410 nm to 530 nm for blue, 470 nm to 600 nm for green, 545 nm to 660 nm for amber, 590 nm to 720 nm for red, and 725 nm to 900 nm for NIR.

FIG. 6B includes a table demonstrating the dominant wavelengths for each color of light shown in the graph in FIG. 6A, including the minimum, maximum, and typical wavelengths for each color. It should be noted that though the table presents rather narrow spectra for each light color, the true range of wavelengths of emission for each color may be greater than presented in the table of FIG. 6B and/or the graph of FIG. 6A.

FIG. 6C provides an example layout of six light emitting sources in a LED cluster 88. Each source of the six light emitting sources may match to one of the six colors of light included in the graph of FIG. 6A. In some embodiments, the inclusion of such a range of colored light sources enables the LED cluster 88 to emit natural light. The LED cluster 88 may emit a greater range of light in the visible spectrum than an embodiment with fewer light emitting sources, as will be discussed. In many embodiments, the light emitting sources are LEDs. The light emitting sources may be different sources of light. In some embodiments, the LED cluster 88 is sized and configured to fit beneath a Fresnel lens 31 such that each light emitting source of the LED cluster 88 is able to emit light through the Fresnel lens 31. The system 10 may be configured to power on/off all the light emitting sources of a particular color across an entire light therapy panel 12 individually or as a group.

A multitude of different treatment options are available with LED clusters 88 comprising six light emitting sources. A user 64 may choose which color(s) of light to emit from each cluster 88, the brightness level, and for how long, for each treatment session. In some embodiments, the clusters 88 in the same light therapy unit 11 are all programmed together, such that customization of light emission extends to the level of each light therapy unit 11, rather than each individual cluster 88. However, the system 10 may be customizable to the level of each individual light emitting source in each individual LED cluster 88. The system 10 may be configured so that there is an even distribution of light emitting sources of each color across each unit 11, pallet 58, and/or panel 12. In some embodiments, emission of a particular color of light from a particular light emitting source is achieved by providing more power to that light emitting source, as well as suppressing the power provided to the other light emitting sources (of the non-desired colors).

FIG. 6D illustrates a graph showing output (in mW/nm) vs. wavelength (in nm) for red and infrared light, according to some embodiments. The outputs for two different forms of red light (630 nm and 660 nm wavelengths) may be about 1570 mW/nm. The output for infrared light at a wavelength of 850 nm may be about 700 mW/nm. In some embodiments, the total light output power is equal to or higher than what is outlined by the graph in FIG. 6D. It should be noted that FIGS. 6D-6F may be considered to represent an embodiment of the system 10 previously shown in FIGS. 4C, 4D, and 5F, where there are only two colors of light emitting sources (red and IR), rather than six colors as shown in FIGS. 6A-6C.

In some embodiments, each light therapy panel 12 includes 200 LEDs. The LEDs may be evenly distributed across each light therapy panel 12, such that each panel 12 includes 100 red LEDs 26 and 100 infrared LEDs 28 evenly dispersed across the treatment surface 32, as shown by the shaded and blank circles in FIGS. 4C and 5F. Each light therapy panel 12 may be configured to power on/off the red and infrared LEDs 26, 28 individually or together. For example, when a user 64 desires treatment of only red light, the system 10 may be configured to only turn on the red LEDs 26 without turning on the infrared LEDs 28, and vice versa if the user 64 desires treatment of only infrared light. The system 10 may also be configured to switch light sources or turn on the previously off source during the course of a treatment session. In some embodiments, the system 10 is configured to support both pulsing and non-pulsing “ON” modes. In the non-pulsing “ON” mode, the LEDs 26, 28 do not exhibit any “flicker”; the red and infrared light is emitted in a steady fashion. In the pulsing “ON” mode, the LEDs may cycle and exhibit some flickering.

In some embodiments, and as represented by FIGS. 6E and 6F, the 100 red LEDs 26 are divided into two groups, wherein each group of red LEDs 26 emits red light of a different wavelength. For example, one group of red LEDs 26 may be LEDs that emit red light of a wavelength of 630 nm (see FIG. 6F), and the other group of red LEDs 26 may be LEDs that emit red light of a wavelength of 660 nm (see FIG. 6E). Similar to the even distribution between red and infrared LEDs 26, 28, the red LEDs may be divided equally such that each light therapy panel 12 includes 50 red LEDs that emit light of 630 nm and 50 red LEDs that emit light of 660 nm, and the red LEDs of different wavelengths are evenly distributed across the treatment surface 32. In many embodiments, the infrared LEDs 28 emit light of a wavelength of 850 nm. In some embodiments, the system 10 is configured to operate the red LEDs of different wavelengths independent of one another, such that only the red LEDs that emit at 630 nm are turned on and the red LEDs that emit at 660 nm are turned off, or vice versa.

FIGS. 7A-7C show top and/or bottom views of a pallet 58. FIG. 7A illustrates a pallet 58 similar to the pallet 58 depicted in FIG. 5D, including first, second, and third heat sinks 6a-c. FIG. 7A also includes seven Fresnel lenses 31, which are shown some distance removed from the heat sinks 60a-c. It should be noted that this distance is intended to demonstrate the location of the light box/lens holder 14 of each light therapy unit 11, which is not included in the figure. The figure does include the housing 13 of the light therapy panel 12. As shown, each of the first, second, and third heat sinks 60a-c may define a slight curvature that contributes to the overall curvature 90 of the pallet 58. In many embodiments, the curvature of each heat sink 60a-c is slight so that each heat sink 60a-c can maintain substantially full contact with each light therapy unit 11 coupled to each heat sink 60a-c. Because the light therapy units 11 have rigid light boxes/lens holders 14, a more dramatic curve of each heat sink 60a-c may not be compatible with mounting a rigid light therapy unit 11 and maintaining substantially full contact of the printed circuit board 56 of each light therapy unit 11 with the curved heat sink 60a-c.

FIG. 7B is similar to FIG. 7A, and shows a top/bottom view of a pallet 58 including seven light therapy units 11a-g. In contrast to FIG. 7A, FIG. 7B includes a single continuous heat plate 61 rather than multiple heat sinks 60a-c. As previously discussed, the heat plate 61 may be configured to mount on an entire pallet 58. Similar to the heat sinks 60, the curvature of the heat plate 61 contributes to the overall curvature 90 of the pallet 58. In some embodiments, as illustrated in FIG. 7B, the heat plate 61 defines a smooth curve. The heat plate 61 may define a less smooth curve that may include slightly sharper curves in the spaces on the plate 61 between light therapy units 11. This shape may help facilitate fitting all seven light therapy units 11a-g on a single heat plate 61 by providing substantially straight, non-curved portions of the heat plate 61 for mounting each light therapy unit 11. Stated differently, the heat plate 61 may appear mostly non-curved where the heat plate 61 is coupled to each light therapy unit 11a-g, and the overall curvature of the heat plate 61 may result from curves in the plate 61 that are located in the spaces between light therapy units 11a-g.

FIG. 7C is similar to FIG. 7B in that it includes a heat plate 61 rather than multiple heat sinks 60. FIG. 7C also shows in greater detail how each light therapy unit 11a, 11b is coupled to the heat plate 61: via a plurality of mounting screws 66. In some embodiments, the light therapy panel 12 includes a back bucket 72. The back bucket 72 may be thought of as a rear cover of the light therapy panel 12. In some embodiments, the heat plate 61 is coupled to the back bucket 72 via mounting screws 66 inserted into the mounting holes 74 shown in FIG. 5E. As illustrated in FIG. 7C, the back bucket 72 may be located some distance behind the heat plate 61. In many embodiments, this distance is intended as a gap for convection and/or forced air cooling of the light therapy panel 12. The gap may include at least one fan 54 (not shown) configured to facilitate cooling of the light therapy panel 12. In some embodiments, the at least one fan is a side blow fan configured to pull air from a back side of the heat plate 61 and blow it out upward, toward the top of the light therapy panel 12. As such, the input and output of heat in a side blow fan may be at a 90-degree angle. The light therapy panel 12 may use any other type of fan in the gap between the heat plate 61 and the back bucket 72 to help facilitate cooling of the panel 12. In some embodiments, the gap is about 10 mm wide. The gap may be more or less than 10 mm wide. The back bucket 72 may be comprised of plastic, such as acrylonitrile butadiene styrene (“ABS”) or polyvinyl chloride (“PVC”) plastic. It should be noted that though FIG. 7C is illustrated to include only three light therapy units 11a-c, it may represent a pallet 58. Though typically depicted with seven light therapy units 11a-g, a pallet 58 can comprise more or fewer than seven light therapy units 11a-g.

FIGS. 8A-8C illustrate top and/or bottom views of the light therapy system 10, including a varying number of light therapy panels 12. FIG. 8A illustrates a first light therapy panel 12a and a second light therapy panel 12b. In some embodiments, when the first light therapy panel 12a is located adjacent the second light therapy panel 12b, the first and second light therapy panels 12a, 12b are configured to curve at least about 90-degrees, as demonstrated by FIG. 8A. While FIG. 8A illustrates a curvature of about 90-degrees, it should be appreciated that the curvature may be configured to any curvature greater than or less than 90-degrees, such as 45-degrees, 60-degrees, 100-degrees, 120-degrees, or 180-degrees.

FIG. 8B shows the system 10 including four light therapy panels 12a-d, and demonstrates that, in some embodiments, when the first light therapy panel 12a is located adjacent the second light therapy panel 12b, the second light therapy panel 12b is located adjacent the third light therapy panel 12c, and the third light therapy panel 12c is located adjacent the fourth light therapy panel 12d, the first, second, third, and fourth light therapy panels 12a-d are configured to curve at least 180-degrees. While FIG. 8B illustrates a curvature of about 180-degrees, it should also be appreciated that the curvature may be configured to any curvature greater than or less than 180-degrees, such as 45-degrees, 60-degrees, 90-degrees, 120-degrees, 150-degrees, 210-degrees, 240-degrees, 270-degrees, or 360-degrees.

FIG. 8C shows a light therapy system 10 including eight light therapy panels 12a-h. As illustrated by FIG. 8C, in some embodiments, when the first light therapy panel 12a is located adjacent the second light therapy panel 12b, the second light therapy panel 12b is located adjacent the third light therapy panel 12c, the third light therapy panel 12c is located adjacent the fourth light therapy panel 12d, the fourth light therapy panel 12d is located adjacent the fifth light therapy panel 12e, the fifth light therapy panel 12e is located adjacent the sixth light therapy panel 12f, the sixth light therapy panel 12f is located adjacent the seventh light therapy panel 12g, and the seventh light therapy panel 12g is located adjacent the eighth light therapy panel 12h, the first, second, third, fourth, fifth, sixth, seventh, and eighth light therapy panels 12a-h are configured to curve at least 360-degrees. The first light therapy panel 12a may also be located adjacent the eighth light therapy panel 12h. Though not included in FIGS. 8A-8C, it should be understood that the treatment surface 32 of each light therapy panel 12a-h is the inner, concave surface of each light therapy panel 12a-h. As such, a user 64 may be positioned, for example, in the middle of the circle in FIG. 8C, in order to receive treatment.

Furthermore, the light therapy panels 12 may be mechanically coupled together via any suitable detachable coupling mechanism. In some embodiments, two or more light therapy panels 12 are detachably coupled together via an H lock key. Moreover, in the embodiment illustrated in FIG. 8C, the light therapy system 10 has a radius of about 61.115 cm. Each light therapy panel 12a-h may have a width of about 48 cm. Each light therapy panel 12a-h may have a height of about 96.8 cm.

FIG. 9A shows a front isometric view of the console hub 18. As previously discussed, the console hub 18 may work as a central controller for the whole light therapy system 10 and may be coupled to each light therapy panel 12. FIGS. 9B and 9C illustrate front and back views, respectively, of the console hub 18. The front view shows that, in some embodiments, the console hub 18 includes a liquid crystal display (“LCD”) touch screen 38 that may act as a user interface configured to receive commands to operate the console hub 18. In some embodiments, the LCD screen 38 is a seven-inch full color, high-resolution display with multi-touch technology. The console hub 18 may also include a plurality of connection ports 36 for panel cables 20a-d. In some embodiments, the console hub 18 has four connection ports 36 to provide connection for up to four light therapy panels 12a-d. In other embodiments, the console hub 18 has one, two, or three connection ports 36. Each connection port 36 may include a power supply providing about 48 Volts at about 200 Watts.

As shown in FIG. 9C, the back of the console hub 18 may include ventilation openings 42 to facilitate air flow through the console, and in conjunction with at least one internal low-noise fan 40 maintain proper working temperature. In some embodiments, the console hub 18 acts as the master controller interfacing with a user and controlling the light therapy system 10. The console hub 18 may have LAN and WLAN connections able to connect to the cloud for various applications included but not limited to downloading updates as required, storing user data (if permitted) and other possible features.

In some embodiments, the back of the console hub 18 has an AC input socket supporting about 90-264 Volts at about 15 Amps. The console hub 18 may be turned off without completely cutting off the AC power supply, and may consume less than 0.5 Watts in the “off” state. In some embodiments, the console hub 18 is turned on and off via an ON/OFF button located on the front of the console hub 18. The ON/OFF button may include dual color LEDs, where a blue color may indicate that the console hub 18 is turned on and an amber color may indicate that the console hub 18 is turned off and/or in standby mode. The console hub 18 may also support voice input and voice feedback through the use of stereo digital microphones and stereo speakers, respectively, as well as in conjunction with the remote control unit 22.

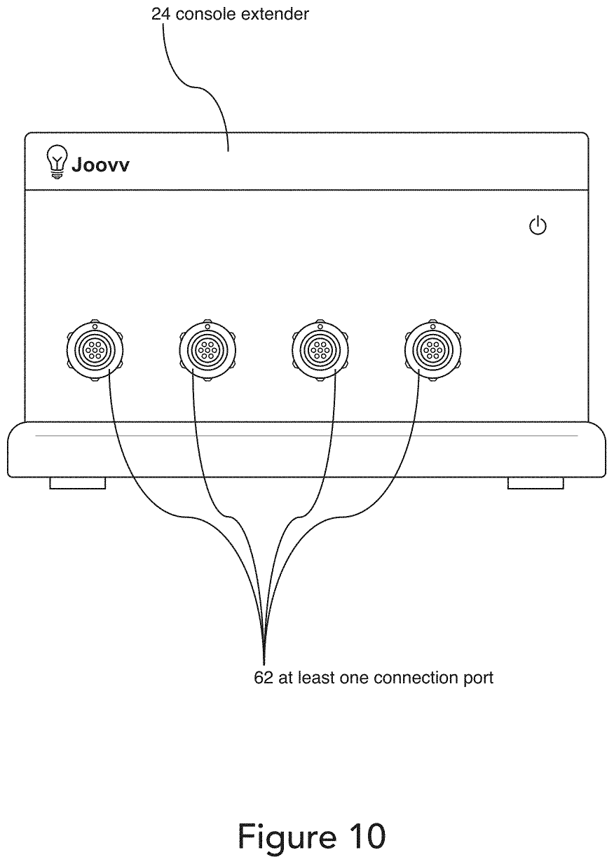

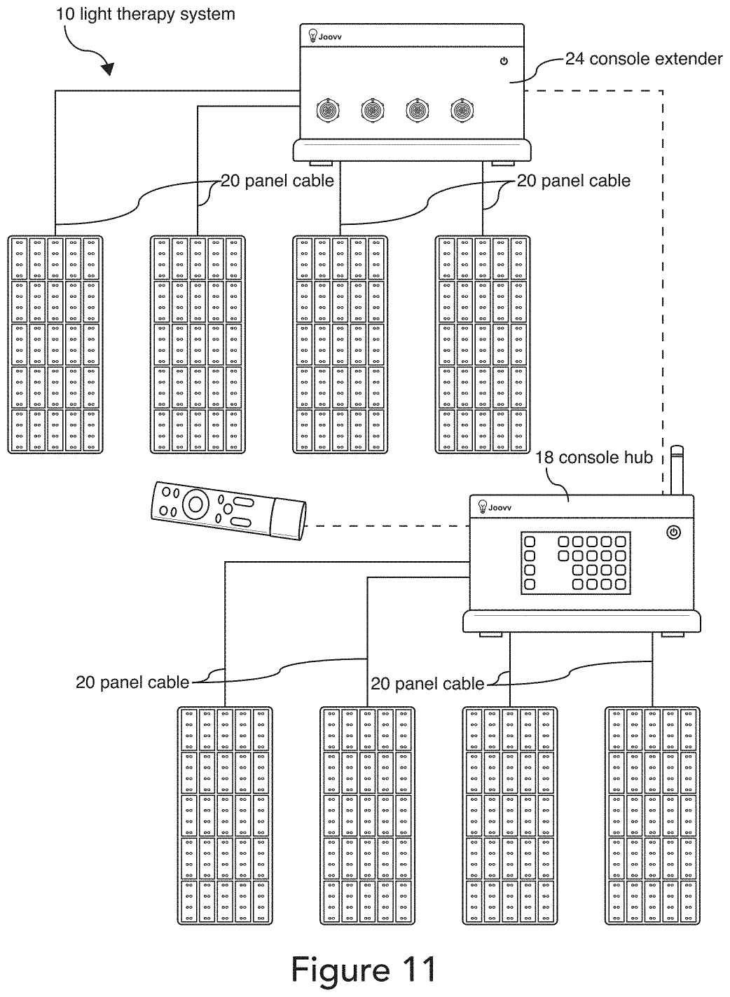

As shown in FIG. 9C, the back of the console hub 18 may include various ports; for example, an Ethernet port, an HDMI port, and a mini-USB port. In some embodiments, the Ethernet port is a one Gigabit Ethernet port with support for energy efficient Ethernet (EEE), Ethernet audio video bridging (AVB), and IEEE 1588 precision time protocol (PTP). An HDMI 2.0a port may be used to support one display up to 1080p, including audio. In some embodiments, a mini-USB port is used to communicatively and electrically couple the console hub 18 to the console hub extender 24, which will be discussed further with reference to FIGS. 10 and 11. In some embodiments, the console hub 18 includes internal antenna(s) configured to enable WiFi and Bluetooth connection over 802.11a/b/g/n/ac and 2.4/5 GHz for WiFi and Bluetooth 4.1. The internal antenna(s) may be located within a plastic housing of the console hub 18. Alternatively, the internal antenna(s) may be located elsewhere on and/or within the housing.

The console hub 18 may use a Coral SOM3 processing engine and support a Debian Linux operating system. Alternatively, the console hub 18 may use any other appropriate processing engine and operating system. The console hub 18 may have a logic board, which may be an AI edge based logic board or a non-AI logic board. In some embodiments, for example when the console hub 18 includes an AI edge based logic board, the processing engine supports artificial intelligence (AI) functionality. Future embodiments and/or updates to current embodiments of the light therapy system 10 may include AI functionality. Future AI functionality may include configuration with a remote camera interface, which may be able to scan and/or map surfaces of the human body in order to detect changes in a user 64 from the effects of the light therapy provided by the system 10.

FIG. 10 illustrates a front view of a console hub extender 24 of a light therapy system 10, according to some embodiments. The hub extender 24 shown in FIG. 10 has four connection ports 62 to allow for the connection of up to four additional light therapy panels 12 to the system 10. The hub extender 24 may comprise fewer or more than four connection ports 62. For example, one embodiment of the console extender 24 includes two connection ports 62. Each connection port 62 may be configured to support one additional light therapy panel 12. In some embodiments, the hub extender 24 is communicatively coupled to the console hub 18 via USB link. The USB link may be a mini-USB link. The hub extender 24 may include an AC power cord in order to plug into an AC wall socket to receive power, but may not include an AC-kill switch. The hub extender 24 may not need the kill switch since the extender 24 is controlled by the console hub 18, and unplugging the USB cable and/or issuing a command from the console hub 18 will turn off the extender 24 and any and all light therapy panels 12 coupled to the extender 24. Similar to the console hub 18, the hub extender 24 may comprise at least one ventilation opening and at least one low noise fan 40 for maintaining proper working temperature. In many embodiments, the hub extender 24 and the console hub 18 use the same type of panel cables 20 to connect to light therapy panels 12. The console hub extender 24 may have an LED located on the front of the unit to indicate the status of the extender 24. In some embodiments, the LED is blue. In some embodiments, the console hub extender 24 has much and/or all of the same functionality of the console hub 18, including the potential for AI functionality, WiFi connection, etc.

FIG. 11 illustrates an extended light therapy system 10, according to some embodiments. FIG. 11 includes a console hub 18 and a console hub extender 24, and shows each of the console hub 18 and console extender 24 coupled to four light therapy panels 12. Accordingly, in such embodiments, eight light therapy panels 12 may be included in a light therapy system 10, as depicted in FIGS. 8C and 11. As previously discussed, the console hub 18 and the console hub extender 24 may be communicatively and electrically coupled via a wireless or wired connection, such as via USB, as shown by the line connecting the console hub 18 and the console extender 24. In some embodiments, the USB connection is a mini-USB connection. Both the hub 18 and the extender 24 may be communicatively and electrically coupled to light therapy panels 12 through panel cables 20. In some embodiments, each panel cable 20 uses a 6-pin power DIN polarized connector for quick connect and disconnect without the need for screws or twisting. In addition, each panel cable 20 may have symmetrical ends such that the cable 20 can connect to the hub extender 24 or light therapy panel 12 from either end. In some embodiments, each panel cable 20 defines a length of up to about 5 meters and a diameter of about 6 mm or less. In many embodiments, each panel cable 20 is fully shielded. In some embodiments, all eight light therapy panels 12 are configured to be positioned in a variety of possible configurations, including a configuration that completely encircles a user 64, as illustrated in FIG. 8C. Multiple users 64 may use the system 10 simultaneously in an extended configuration, or in a more standard configuration of one to four light therapy panels 12.

While FIG. 11 illustrates an extended light therapy system 10 having eight light therapy panels 12, it should be appreciated that the system 10 may be configured with any number of light therapy panels 12 greater than or less than eight. In some embodiments, the system 10 includes five, six, seven, nine, ten, twelve, twenty, or more light therapy panels 12. The light therapy system 10 may be arranged according to a variety of configurations as desired by the user.

None of the steps described herein is essential or indispensable. Any of the steps can be adjusted or modified. Other or additional steps can be used. Any portion of any of the steps, processes, structures, and/or devices disclosed or illustrated in one embodiment, flowchart, or example in this specification can be combined or used with or instead of any other portion of any of the steps, processes, structures, and/or devices disclosed or illustrated in a different embodiment, flowchart, or example. The embodiments and examples provided herein are not intended to be discrete and separate from each other.

The section headings and subheadings provided herein are nonlimiting. The section headings and subheadings do not represent or limit the full scope of the embodiments described in the sections to which the headings and subheadings pertain. For example, a section titled “Topic 1” may include embodiments that do not pertain to Topic 1 and embodiments described in other sections may apply to and be combined with embodiments described within the “Topic 1” section.

Some of the devices, systems, embodiments, and processes use computers. Each of the routines, processes, methods, and algorithms described in the preceding sections may be embodied in, and fully or partially automated by, code modules executed by one or more computers, computer processors, or machines configured to execute computer instructions. The code modules may be stored on any type of non-transitory computer-readable storage medium or tangible computer storage device, such as hard drives, solid state memory, flash memory, optical disc, and/or the like. The processes and algorithms may be implemented partially or wholly in application-specific circuitry. The results of the disclosed processes and process steps may be stored, persistently or otherwise, in any type of non-transitory computer storage such as, e.g., volatile or non-volatile storage.

The various features and processes described above may be used independently of one another, or may be combined in various ways. All possible combinations and subcombinations are intended to fall within the scope of this disclosure. In addition, certain method, event, state, or process blocks may be omitted in some implementations. The methods, steps, and processes described herein are also not limited to any particular sequence, and the blocks, steps, or states relating thereto can be performed in other sequences that are appropriate. For example, described tasks or events may be performed in an order other than the order specifically disclosed. Multiple steps may be combined in a single block or state. The example tasks or events may be performed in serial, in parallel, or in some other manner. Tasks or events may be added to or removed from the disclosed example embodiments. The example systems and components described herein may be configured differently than described. For example, elements may be added to, removed from, or rearranged compared to the disclosed example embodiments.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require at least one of X, at least one of Y, and at least one of Z to each be present.

The term “and/or” means that “and” applies to some embodiments and “or” applies to some embodiments. Thus, A, B, and/or C can be replaced with A, B, and C written in one sentence and A, B, or C written in another sentence. A, B, and/or C means that some embodiments can include A and B, some embodiments can include A and C, some embodiments can include B and C, some embodiments can only include A, some embodiments can include only B, some embodiments can include only C, and some embodiments can include A, B, and C. The term “and/or” is used to avoid unnecessary redundancy.

The term “about” is used to mean “approximately” with reference to numerical values. More specifically, “about” and “approximately” may be understood to mean the stated value +/−10%. For example, a light therapy panel that measures “about” 12″ by 36″ may measure anywhere between or including 10.8″ by 39.6.″ In another example, at least one LED cluster comprising a first light emitting source capable of emitting royal blue light within a wavelength of “approximately” 380 nm to 505 nm means that at least one LED cluster comprising a first light emitting source capable of emitting royal blue light within any wavelength between 342 nm to 550 nm.

The term “substantially” is used to mean “completely” or “nearly completely”. For example, the disclosure includes, “the plurality of lights are substantially evenly distributed across each light therapy panel 12a-d”. In this context, “substantially evenly distributed” is used to mean “completely” or “nearly completely” evenly distributed. As such, the plurality of lights may be slightly unevenly distributed and fall into the threshold of “substantially evenly” distributed.

While certain example embodiments have been described, these embodiments have been presented by way of example only, and are not intended to limit the scope of the inventions disclosed herein. Thus, nothing in the foregoing description is intended to imply that any particular feature, characteristic, step, module, or block is necessary or indispensable. Indeed, the novel methods and systems described herein may be embodied in a variety of other forms; furthermore, various omissions, substitutions, and changes in the form of the methods and systems described herein may be made without departing from the spirit of the inventions disclosed herein.

1. A light therapy system, comprising:
a rigid curved housing defining a concave curvature;
a plurality of light therapy panels, wherein each light therapy panel of the plurality of light therapy panels comprises a plurality of lights arranged and configured to emit at least one of red light and infrared light;
a console hub communicatively and electrically coupled to the plurality of light therapy panels, the console hub configured to operate the plurality of light therapy panels; and
a remote control unit communicatively coupled to the console hub, wherein the remote control unit is configured to operate at least one of the console hub and the plurality of light therapy panels.

2. The light therapy system of claim 1, wherein a first light therapy panel of the plurality of light therapy panels comprises at least one light therapy unit and at least one light therapy pallet;
wherein each light therapy unit of the at least one light therapy unit is elongate along a first direction; and
wherein each light therapy pallet of the at least one light therapy pallet extends along a second direction that is perpendicular to the first direction.

3. The light therapy system of claim 2, wherein a first light therapy pallet of the at least one light therapy pallet comprises a first light therapy unit, a second light therapy unit, and a third light therapy unit aligned along the second direction;
wherein the first light therapy unit is located between the second light therapy unit and the third light therapy unit; and
wherein the first light therapy unit, the second light therapy unit, and the third light therapy unit are electrically coupled.

4. The light therapy system of claim 3, wherein the rigid curved housing defines a concave curvature such that the first light therapy pallet curves along a third direction that is perpendicular to both the first direction and the second direction.

5. The light therapy system of claim 3, further comprising a first heat sink mechanically coupled to the first light therapy unit, the second light therapy unit, and the third light therapy unit, wherein the first heat sink is at least partially located within the rigid curved housing.

6. The light therapy system of claim 5, further comprising a fourth light therapy unit, a fifth light therapy unit, a sixth light therapy unit, a seventh light therapy unit, a second heat sink, and a third heat sink, wherein the second heat sink is mechanically coupled to the fourth light therapy unit and the sixth light therapy unit and the third heat sink is mechanically coupled to the fifth light therapy unit and the seventh light therapy unit, and wherein the second heat sink and the third heat sink are at least partially located within the rigid curved housing.

7. The light therapy system of claim 3, wherein the first light therapy unit comprises a controller board and a board-to-board connector capable of electrically and communicatively coupling with another controller board.

8. The light therapy system of claim 2, wherein the first light therapy panel comprises at least seven light therapy units, wherein the at least seven light therapy units are aligned along at least one of the first direction and the second direction.

9. The light therapy system of claim 2, wherein the first light therapy panel comprises at least five light therapy pallets, wherein the at least five light therapy pallets are aligned along the first direction.

10. The light therapy system of claim 2, wherein each light therapy unit of the at least one light therapy unit comprises at least one LED cluster comprising six or more light emitting sources.

11. The light therapy system of claim 10, wherein the at least one LED cluster comprises:
a first light emitting source capable of emitting royal blue light within a wavelength of approximately 380 nm to 505 nm,
a second light emitting source capable of emitting blue light within a wavelength of approximately 410 nm to 530 nm,
a third light emitting source capable of emitting green light within a wavelength of approximately 470 nm to 600 nm,
a fourth light emitting source capable of emitting amber light within a wavelength of approximately 545 nm to 660 nm,
a fifth light emitting source capable of emitting red light within a wavelength of approximately 590 nm to 720 nm; and
a sixth light emitting source capable of emitting NIR light within a wavelength of approximately 725 nm to 900 nm.

12. The light therapy system of claim 10, wherein each light therapy unit of the at least one light therapy unit comprises a first LED cluster, a second LED cluster, a third LED cluster, and a fourth LED cluster, wherein each of the first LED cluster, the second LED cluster, the third LED cluster, and the fourth LED cluster are aligned along the first direction.

13. The light therapy system of claim 10, wherein each light therapy unit of the at least one light therapy unit further comprises a lens located over a single LED cluster of the at least one LED cluster, wherein the lens comprises a Fresnel lens.

14. The light therapy system of claim 10, further comprising four LED clusters and four Fresnel lenses, such that each LED cluster of the four LED clusters is configured to emit light through a single Fresnel lens of the four Fresnel lenses.

15. The light therapy system of claim 1, wherein the console hub further comprises a liquid crystal display (LCD) touch screen arranged and configured to receive commands to operate the console hub.

16. The light therapy system of claim 2, further comprising a second light therapy panel communicatively and electrically coupled to the console hub, the second light therapy panel comprising a rigid curved housing defining a concave curvature and a second plurality of lights arranged and configured to emit at least one of red light and infrared light.

17. The light therapy system of claim 16, wherein when the first light therapy panel is located adjacent the second light therapy panel, the rigid curved housing of the first light therapy panel and the rigid curved housing of the second light therapy panel are configured such that the concave curvature is at least 90-degrees.

18. The light therapy system of claim 16, further comprising a third light therapy panel communicatively and electrically coupled to the console hub, the third light therapy panel comprising a rigid curved housing defining a concave curvature and a third plurality of lights arranged and configured to emit at least one of red light and infrared light, and a fourth light therapy panel communicatively and electrically coupled to the console hub, the fourth light therapy panel comprising a rigid curved housing defining a concave curvature and a fourth plurality of lights arranged and configured to emit at least one of red light and infrared light.

19. The light therapy system of claim 18, wherein when the first light therapy panel is located adjacent the second light therapy panel, the second light therapy panel is located adjacent the third light therapy panel, and the third light therapy panel is located adjacent the fourth light therapy panel, the rigid curved housing of the first light therapy panel, the rigid curved housing of the second light therapy panel, the rigid curved housing of the third light therapy panel, and the rigid curved housing of the fourth light therapy panel are configured such that the concave curvature is at least 180-degrees.

20. The light therapy system of claim 1, further comprising a console hub extender communicatively and electrically coupled to the console hub and at least one additional light therapy panel, such that the console hub extender enables inclusion of at least five light therapy panels in the light therapy system.
